(19) United States
(12) Patent Application Publication
(10) Pub. No.: US 2024/0043553 A1
(43) Pub. Date: Feb. 8, 2024

(54) METHODS FOR TREATMENT OF PREVIOUSLY UNTREATED FOLLICULAR LYMPHOMA WITH MOSUNETUZUMAB AND LENALIDOMIDE

(71) Applicants: Genentech, Inc., South San Francisco, CA (US); Hoffmann-La Roche Inc., Little Falls, NJ (US)

(72) Inventors: Andrea KNAPP, Basel (CH); Chi-Chung LI, South San Francisco, CA (US); Enkhtsetseg PUREV, South San Francisco, CA (US); Michael C. WEI, South San Francisco, CA (US)

(21) Appl. No.: 18/338,985

(22) Filed: Jun. 21, 2023

Related U.S. Application Data
(60) Provisional application No. 63/354,491, filed on Jun. 22, 2022.

(51) Int. Cl. C07K 16/28; A61K 31/4412; A61K 31/573; A61K 45/06; A61P 35/00

(52) U.S. Cl. CPC C07K 16/2887; C07K 16/2809; A61K 31/4412; A61K 31/573; A61K 45/06; A61P 35/00; A61K 2039/505

(57) ABSTRACT

The present invention relates to the treatment of subjects having previously untreated follicular lymphoma (FL). More specifically, the invention pertains to the treatment of subjects having previously untreated FL by administering a combination of mosunetuzumab and lenalidomide.

### SEQUENCE LISTING

The instant application contains a Sequence Listing which has been submitted electronically in XML format and is hereby incorporated by reference in its entirety. Said XML copy, created on Jun. 2, 2023, is named 50474-298002_Sequence_Listing_6_2_23 and is 34,004 bytes in size.

### FIELD OF THE INVENTION

The present invention relates to the treatment of subjects having previously untreated follicular lymphoma (FL). More specifically, the invention pertains to combination treatment of subjects having previously untreated FL by administration of mosunetuzumab and lenalidomide.

### BACKGROUND

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. Indolent cancers can also severely effect quality of life. Cancer care thus represents a significant and ever-increasing societal burden.

B cell proliferative disorders are a leading cause of cancer-related deaths. For example, non-Hodgkin's lymphoma (NHL) advances quickly and is fatal if untreated. Lymphomas of B-cell origin constitute a diverse set of neoplasms within the larger context of non-Hodgkin lymphoma (NHL). Follicular lymphoma (FL) is the most common subtype of indolent NHL (Al-Hamandi et al. 2015). There is no standard treatment for the initial management of FL and questions regarding the optimal anti-CD20 monoclonal antibody, the optimal chemotherapy backbone and additional maintenance treatment remains still unclear. Despite the encouraging response to currently available immunotherapy based first-line therapies, most patients will eventually relapse. Relapses are characterized by increasing refractoriness and several important needs remain unmet, therapies are not yet curative and the majority of patients will experience progression of the disease. Therefore, there is need for novel therapies to increase the anti-tumor activity and to prolong remission in patients with previously untreated FL.

### SUMMARY OF THE INVENTION

The present invention relates to methods of treating a subject having a previously untreated follicular lymphoma (FL) by administration of mosunetuzumab and lenalidomide as a combination therapy. In particular, the present invention relates to methods of treating a subject having a previously untreated FL by subcutaneous administration of mosunetuzumab and oral administration of lenalidomide.

In one aspect, the invention features a method of treating a subject having a previously untreated follicular lymphoma (FL), the method comprising administering to the subject mosunetuzumab and lenalidomide according to a dosing regimen comprising a first 21-day (±1 day) dosing cycle and a second 28-day (±1 day) dosing cycle, wherein: (a) the first dosing cycle comprises a first dose (C1D1), a second dose (C1D2), and a third dose (C1D3) of mosunetuzumab subcutaneously administered on Days 1, 8 (±1 day), and 15 (±1 day), respectively, of the first dosing cycle, wherein the C1D1 is about 5 mg (e.g., 5 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, or ±1 mg; e.g., 5 mg), the C1D2 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and the C1D3 is about mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), (b) the second dosing cycle comprises a single dose (C2D1) of mosunetuzumab subcutaneously administered on Day 1 of the second dosing cycle, wherein the C2D1 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and (c) the second dosing cycle further comprises orally administering between about 5 mg and about 20 mg (e.g., between about 5 mg and about 10 mg, between about 10 mg and about 15 mg, between about 15 mg and about 20 mg, or between about 5 mg and about mg; e.g., about 5, 6, 7, 8, 9, 10, 11, 12, 13, 14, 15, 16, 17, 18, 19, or 20 mg) lenalidomide daily on Days 1-21 of the second dosing cycle. In some embodiments, the first dosing cycle is a three-dose dosing cycle (i.e., comprising three doses of mosunetuzumab).

In some embodiments, the dosing regimen comprises one or more (e.g., 1, 2, 3, 4, 5, 6, 7, 8, 9, 10, or more) additional dosing cycles. In some embodiments, the dosing regimen comprises one to ten (e.g., 1, 2, 3, 4, 5, 6, 7, 8, 9, or 10) additional dosing cycles. In some embodiments, the dosing regimen comprises ten additional dosing cycles. In some embodiments, the length of each of the one or more additional dosing cycles is about 28 days (±1 day).

In some embodiments, each of the one or more additional dosing cycles comprises an additional single dose of mosunetuzumab. In some embodiments, the method comprises subcutaneously administering to the subject each additional single dose of mosunetuzumab on Day 1 of each of the one or more additional dosing cycles. In some embodiments, the additional single dose of mosunetuzumab is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg). In some embodiments, each additional single dose of mosunetuzumab is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg).

In some embodiments, lenalidomide is not administered during the first dosing cycle. In some embodiments, lenalidomide is orally administered during any of the one or more (e.g., 1, 2, 3, 4, 5, 6, 7, 8, 9, 10, or more) additional dosing cycles. In some embodiments, lenalidomide is orally administered during each of the ten additional dosing cycles. In some embodiments, lenalidomide is orally administered on Days 1-21 of each of the additional dosing cycles comprising administration of lenalidomide. In some embodiments, lenalidomide is not administered on the last 7 days (±1 day) of any dosing cycle comprising administration of lenalidomide. In some embodiments, lenalidomide is administered at a dose of about 10 mg (e.g., 10 mg±0.025 mg, ±0.05 mg, ±0.075 mg, ±0.1 mg, ±0.2 mg, ±0.3 mg, ±0.4 mg, ±0.5 mg, ±0.75 mg, ±1 mg, or ±2 mg; e.g., 10 mg). In some embodiments, lenalidomide is administered at a dose of about 20 mg (e.g., 20 mg±0.05 mg, ±0.1 mg, ±0.2 mg, ±0.3 mg, ±0.4 mg, ±0.5 mg, ±0.75 mg, ±1 mg, ±1.5 mg, ±2 mg, ±3 mg, or ±4 mg; e.g., 20 mg).

In one aspect, the invention features a method of treating a subject having a previously untreated FL, the method comprising administering to the subject mosunetuzumab and lenalidomide according to a dosing regimen comprising a first 21-day (±1 day) dosing cycle and a second 28-day (±1 day) dosing cycle, wherein: (a) the first dosing cycle comprises a first dose (C1D1), a second dose (C1D2), and a third dose (C1D3) of mosunetuzumab subcutaneously administered on Days 1, 8 (±1 day), and 15 (±1 day), respectively, of the first dosing cycle, wherein the C1D1 is about 5 mg (e.g., 5 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, or ±1 mg; e.g., 5 mg), the C1D2 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and the C1D3 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), (b) the second dosing cycle comprises a single dose (C2D1) of mosunetuzumab subcutaneously administered on Day 1 of the second dosing cycle, wherein the C2D1 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and (c) the second dosing cycle further comprises orally administering about 10 mg (e.g., 10 mg±0.025 mg, ±0.05 mg, ±0.075 mg, ±0.1 mg, ±0.2 mg, ±0.3 mg, ±0.4 mg, ±0.5 mg, ±0.75 mg, ±1 mg, or ±2 mg; e.g., 10 mg) lenalidomide daily on Days 1-21 of the second dosing cycle.

In one aspect, the invention features a method of treating a subject having a previously untreated FL, the method comprising administering to the subject mosunetuzumab and lenalidomide according to a dosing regimen comprising a first 21-day (±1 day) dosing cycle and a second 28-day (±1 day) dosing cycle, wherein: (a) the first dosing cycle comprises a first dose (C1D1), a second dose (C1D2), and a third dose (C1D3) of mosunetuzumab subcutaneously administered on Days 1, 8 (±1 day), and 15 (±1 day), respectively, of the first dosing cycle, wherein the C1D1 is about 5 mg (e.g., 5 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, or ±1 mg; e.g., 5 mg), the C1D2 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and the C1D3 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), (b) the second dosing cycle comprises a single dose (C2D1) of mosunetuzumab subcutaneously administered on Day 1 of the second dosing cycle, wherein the C2D1 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and (c) the second dosing cycle further comprises orally administering about 20 mg (e.g., 20 mg±0.05 mg, ±0.1 mg, ±0.2 mg, ±0.3 mg, ±0.4 mg, ±0.5 mg, ±0.75 mg, ±1 mg, ±1.5 mg, ±2 mg, ±3 mg, or ±4 mg; e.g., 20 mg) lenalidomide daily on Days 1-21 of the second dosing cycle.

In one aspect, the invention features a method of treating a subject having a previously untreated FL, the method comprising administering to the subject mosunetuzumab and lenalidomide according to a dosing regimen comprising a first 21-day (±1 day) dosing cycle and eleven subsequent 28-day (±1 day) dosing cycles, wherein: (a) the first dosing cycle comprises a first dose (C1D1), a second dose (C1D2), and a third dose (C1D3) of mosunetuzumab subcutaneously administered on Days 1, 8 (±1 day), and 15 (±1 day), respectively, of the first dosing cycle, wherein the C1D1 is about 5 mg (e.g., 5 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, or ±1 mg; e.g., 5 mg), the C1D2 is about 45 mg (e.g., 45 mg 0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and the C1D3 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), (b) the second to twelfth dosing cycles each comprises a single dose (C2D1-C12D1) of mosunetuzumab subcutaneously administered on Day 1 of each dosing cycle, wherein each single dose C2D1-C12D1 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and (c) the second to twelfth dosing cycles each further comprises orally administering about 10 mg (e.g., 10 mg±0.025 mg, ±0.05 mg, ±0.075 mg, ±0.1 mg, ±0.2 mg, ±0.3 mg, ±0.4 mg, ±0.5 mg, ±0.75 mg, ±1 mg, or ±2 mg; e.g., 10 mg) lenalidomide daily on Days 1-21 of each dosing cycle.

In one aspect, the invention features a method of treating a subject having a previously untreated FL, the method comprising administering to the subject mosunetuzumab and lenalidomide according to a dosing regimen comprising a first 21-day (±1 day) dosing cycle and eleven subsequent 28-day (±1 day) dosing cycles, wherein: (a) the first dosing cycle comprises a first dose (C1D1), a second dose (C1D2), and a third dose (C1D3) of mosunetuzumab subcutaneously administered on Days 1, 8 (±1 day), and 15 (±1 day), respectively, of the first dosing cycle, wherein the C1D1 is about 5 mg (e.g., 5 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, or ±1 mg; e.g., 5 mg), the C1D2 is about 45 mg (e.g., 45 mg 0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and the C1D3 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), (b) the second to twelfth dosing cycles each comprises a single dose (C2D1-C12D1) of mosunetuzumab subcutaneously administered on Day 1 of each dosing cycle, wherein each single dose C2D1-C12D1 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and (c) the second to twelfth dosing cycles each further comprises orally administering about 20 mg (e.g., 20 mg±0.05 mg, ±0.1 mg, ±0.2 mg, ±0.3 mg, ±0.4 mg, ±0.5 mg, ±0.75 mg, ±1 mg, ±1.5 mg, ±2 mg, ±3 mg, or ±4 mg; e.g., 20 mg) lenalidomide daily on Days 1-21 of each dosing cycle.

In some embodiments, the FL is histologically documented as Grade 1, 2, or 3a, but not 3b according to the World Health Organization classification of lymphoid neoplasms (as referenced in Swerdlow S H, et al. Blood 2016; 127:2375-90).

In some embodiments, the subject has been previously determined based on the Groupe d'Etude des Lymphomes Folliculaires (GELF) criteria (Brice et al. J Clin Oncol. 15(3):1110-1117, 1997) to be in need of systemic therapy to treat the previously untreated FL.

In some embodiments, the first dosing cycle further comprises administration of a corticosteroid. In some embodiments, the second dosing cycle further comprises administration of a corticosteroid. In some embodiments, any of the one or more additional dosing cycles comprises administration of a corticosteroid. In some embodiments, a single dose of the corticosteroid is administered to the subject prior to the administration of any dose of mosunetuzumab. In some embodiments, the corticosteroid comprises dexamethasone or methylprednisolone. In some embodiments, the corticosteroid is administered intravenously or orally. In some embodiments, the corticosteroid comprises dexamethasone and is administered at a dose of about 20 mg (e.g., 20 mg±0.05 mg, ±0.1 mg, ±0.2 mg, ±0.3 mg, ±0.4 mg, ±0.5 mg, ±0.75 mg, ±1 mg, ±1.5 mg, ±2 mg, ±3 mg, or ±4 mg; e.g., 20 mg). In some embodiments, the corticosteroid comprises methylprednisolone and is administered at a dose of about 80 mg (e.g., 80 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, ±10 mg, ±12 mg, ±14 mg, or ±16 mg; e.g., 80 mg).

In some embodiments, the first dosing cycle further comprises administration of an antihistamine. In some embodiments, the second dosing cycle further comprises administration of an antihistamine. In some embodiments, any of the one or more additional dosing cycles comprises administration of an antihistamine. In some embodiments, a single dose of the antihistamine is administered to the subject prior to (e.g., 30, 35, 40, 45, 50, 60 70, 80, 90, 120, 150, 180 minutes, or more prior to) the administration of any dose of mosunetuzumab. In some embodiments, the antihistamine is administered to the subject at least 30 minutes (e.g., 30, 35, 40, 45, 50, 60 70, 80, 90, 120, 150, 180 minutes, or more) prior to the administration of any dose of mosunetuzumab. In some embodiments, the antihistamine is administered orally or intravenously. In some embodiments, the antihistamine comprises diphenhydramine hydrochloride and is administered at a dose of about 50-100 mg (e.g., 50-90 mg, 50-80 mg, 50-70 mg, 50-60 mg, 60-100 mg, 70-100 mg, 80-100 mg, 90-100 mg, 70-mg, 60-90 mg, 60-80 mg, 70-90 mg, or 65-85 mg; e.g., about 50 mg, about 55 mg, about 60 mg, about mg, about 70 mg, about 75 mg, about 80 mg, about 85 mg, about 90 mg, about 95 mg, or about 100 mg).

In some embodiments, the first dosing cycle further comprises administration of an anti-pyretic. In some embodiments, the second dosing cycle further comprises administration of an anti-pyretic. In some embodiments, any of the one or more additional dosing cycles comprises administration of an anti-pyretic. In some embodiments, a single dose of the anti-pyretic is administered to the subject prior to the administration of any dose of mosunetuzumab. In some embodiments, the anti-pyretic is administered to the subject at least 30 minutes (e.g., 30, 35, 40, 45, 50, 60 70, 80, 90, 120, 150, 180 minutes, or more) prior to the administration of any dose of mosunetuzumab. In some embodiments, the anti-pyretic is administered orally. In some embodiments, the anti-pyretic comprises acetaminophen and is administered at a dose of about 500-1000 mg (e.g., 500-900 mg, 500-800 mg, 500-700 mg, 500-600 mg, 600-1000 mg, 700-1000 mg, 800-1000 mg, 900-1000 mg, 700-800 mg, 600-900 mg, 600-800 mg, 700-900 mg, or 650-850 mg; e.g., about 500 mg, about 550 mg, about 600 mg, about 650 mg, about 700 mg, about 750 mg, about 800 mg, about 850 mg, about 900 mg, about 950 mg, or about 1000 mg).

In some embodiments, the first dosing cycle further comprises administration of an initial dose of a prophylactic agent against tumor lysis syndrome (TLS). In some embodiments, the second dosing cycle further comprises administration of an initial dose of a prophylactic agent against TLS. In some embodiments, any of the one or more additional dosing cycles comprises administration of an initial dose of a prophylactic agent against TLS. In some embodiments, the initial dose of the prophylactic agent against TLS is administered to the subject prior to the administration of any dose of mosunetuzumab. In some embodiments, the prophylactic agent against TLS comprises allopurinol. In some embodiments, the initial dose of allopurinol is administered about 72 hours (e.g., 72±0.5 hours, ±1 hours, ±2 hours, ±3 hours, ±4 hours, ±8 hours, ±12 hours, or ±16 hours; e.g., 72 hours) prior to the administration of any dose of mosunetuzumab. In some embodiments, additional single doses of allopurinol are administered daily for 6-10 days (±1 day) after the administration of the initial dose. In some embodiments, the initial dose of allopurinol is about 300 mg (e.g., 300 mg±5 mg, ±10 mg, ±15 mg, ±20 mg, ±25 mg, ±30 mg, ±45 mg, or ±60 mg; e.g., 300 mg). In some embodiments, each additional single dose of allopurinol is about 300 mg (e.g., 300 mg±5 mg, ±10 mg, ±15 mg, ±20 mg, ±25 mg, ±30 mg, ±45 mg, or ±60 mg; e.g., 300 mg). In some embodiments, allopurinol is administered orally. In some embodiments, the prophylactic agent against TLS comprises rasburicase. In some embodiments, the initial dose of rasburicase is administered about 30 minutes (e.g., 30±0.5 minutes, 1 minutes, ±2 minutes, ±3 minutes, ±4 minutes, ±5 minutes, or ±6 minutes; e.g., 30 minutes) prior to the administration of any dose of mosunetuzumab. In some embodiments, additional single doses of rasburicase are administered daily for 1-5 days (±1 day) after the administration of the initial dose. In some embodiments, the initial dose of rasburicase is about 0.2 mg/kg (e.g., 0.2±0.005 mg/kg, ±0.01 mg/kg, ±0.02 mg/kg, ±0.03 mg/kg, or ±0.04 mg/kg; e.g., 0.2 mg/kg). In some embodiments, each additional single dose of rasburicase is about 0.2 mg/kg (e.g., 0.2±0.005 mg/kg, ±0.01 mg/kg, ±0.02 mg/kg, ±0.03 mg/kg, or ±0.04 mg/kg; e.g., 0.2 mg/kg). In some embodiments, rasburicase is administered intravenously.

### BRIEF DESCRIPTION OF THE DRAWING

FIG. 1 is a schematic of the study design described in Example 1 for the dosing regimen of the combination therapy of mosunetuzumab and lenalidomide. Mosunetuzumab is administered subcutaneously via injection and lenalidomide is administered orally. Len=lenalidomide; Cycle=dosing cycle.

### DETAILED DESCRIPTION

The present invention relates to methods of treating a subject having a previously untreated follicular lymphoma (FL) by administration of mosunetuzumab and lenalidomide as a combination therapy. Methods described herein comprise subcutaneously administering mosunetuzumab and orally administering lenalidomide to the subject according to a dosing regimen comprising at least a first dosing cycle and a second dosing cycle, wherein: (a) the first dosing cycle comprises a first dose (C1D1), a second dose (C1D2), and a third dose (C1D3) of mosunetuzumab, (b) the second dosing cycle comprises a first dose (C2D1) of mosunetuzumab and daily doses of lenalidomide. In particular, the first dosing cycle is a 21-day (±1 day) dosing cycle and the C1D1, C1D2, and C1D3 doses are administered on or about Days 1, 8 (±1 day), and 15 (±1 day), respectively, and the second dosing cycle is a 28-day (±1 day) dosing cycle and the C2D1 is administered on Day 1. In addition, lenalidomide is administered on Days 1-21 of the second dosing cycle. Methods of the invention additionally include administration of additional therapeutic agents, such as premedication (e.g., with a corticosteroid, an antihistamine, an anti-pyretic, or a prophylactic agent against tumor lysis syndrome (TLS)).

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J.B. Lippincott Company, 1993).

### II. Definitions

It is to be understood that aspects and embodiments of the invention described herein include “comprising,” “consisting,” and “consisting essentially of” aspects and embodiments.

As used herein, the singular form “a,” “an,” and “the” includes plural references unless indicated otherwise.

The term “about” as used herein refers to the usual error range for the respective value readily known to the skilled person in this technical field. Reference to “about” a value or parameter herein includes (and describes) embodiments that are directed to that value or parameter per se. In some embodiments, the term “about” a value or parameter refers to that value or parameter ±10%.

A “disorder” is any condition that would benefit from treatment including, but not limited to, chronic and acute disorders or diseases including those pathological conditions which predispose the mammal to the disorder in question.

The terms “cell proliferative disorder” and “proliferative disorder” refer to disorders that are associated with some degree of abnormal cell proliferation. In one embodiment, the cell proliferative disorder is cancer. In another embodiment, the cell proliferative disorder is a tumor.

The terms “cancer” and “cancerous” refer to or describe the physiological condition in mammals that is typically characterized by unregulated cell growth. Examples of cancer include, but are not limited to, hematologic cancers, such as mature B cell cancers, excluding Hodgkin's lymphoma, but including non-Hodgkin's lymphoma (NHL), such as diffuse large B cell lymphoma (DLBCL), which may be a Richter's transformation. Other specific examples of cancer also include germinal-center B cell-like (GCB) diffuse large B cell lymphoma (DLBCL), activated B cell-like (ABC) DLBCL, follicular lymphoma (FL), transformed FL, mantle cell lymphoma (MCL), acute myeloid leukemia (AML), chronic lymphoid leukemia (CLL), marginal zone lymphoma (MZL), transformed MZL, high grade B-cell lymphoma, primary mediastinal (thymic) large B cell lymphoma (PMLBCL), small lymphocytic leukemia (SLL), lymphoplasmacytic lymphoma (LL), transformed LL, Waldenstrom macroglobulinemia (WM), central nervous system lymphoma (CNSL), Burkitt's lymphoma (BL), B cell prolymphocytic leukemia, splenic marginal zone lymphoma, hairy cell leukemia, splenic lymphoma/leukemia, unclassifiable, splenic diffuse red pulp small B cell lymphoma, hairy cell leukemia variant, heavy chain diseases, α heavy chain disease, γ heavy chain disease, μ heavy chain disease, plasma cell myeloma, solitary plasmacytoma of bone, extraosseous plasmacytoma, extranodal marginal zone lymphoma of mucosa-associated lymphoid tissue (MALT lymphoma), nodal marginal zone lymphoma, pediatric nodal marginal zone lymphoma, pediatric follicular lymphoma, primary cutaneous follicle center lymphoma, T cell/histiocyte rich large B cell lymphoma, primary DLBCL of the CNS, primary cutaneous DLBCL, leg type, EBV-positive DLBCL of the elderly, DLBCL associated with chronic inflammation, lymphomatoid granulomatosis, intravascular large B cell lymphoma, ALK-positive large B cell lymphoma, plasmablastic lymphoma, large B cell lymphoma arising in HHV8-associated multicentric Castleman disease, primary effusion lymphoma: B cell lymphoma, unclassifiable, with features intermediate between DLBCL and Burkitt lymphoma, and B cell lymphoma, unclassifiable, with features intermediate between DLBCL and classical Hodgkin's lymphoma. Further examples of cancer include, but are not limited to, carcinoma, lymphoma, blastoma, sarcoma, and leukemia or lymphoid malignancies, including B cell lymphomas. More particular examples of such cancers include, but are not limited to, multiple myeloma (MM); low-grade/follicular NHL; small lymphocytic (SL) NHL; intermediate-grade/follicular NHL; intermediate-grade diffuse NHL; high-grade immunoblastic NHL; high-grade lymphoblastic NHL; high-grade small non-cleaved cell NHL; bulky disease NHL; AIDS-related lymphoma; and acute lymphoblastic leukemia (ALL); chronic myeloblastic leukemia; and post-transplant lymphoproliferative disorder (PTLD).

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The terms “B cell proliferative disorder” or “B cell malignancy” refer to disorders that are associated with some degree of abnormal B cell proliferation and include, for example, lymphomas, leukemias, myelomas, and myelodysplastic syndromes. In some instances, the B cell proliferative disorder is a lymphoma, such as non-Hodgkin's lymphoma (NHL), including, for example, follicular lymphoma (FL) (e.g., a previously untreated FL). In a particular embodiment, a subject having a previously untreated FL has been determined based on the Groupe d'Etude des Lymphomes Folliculaires (GELF) criteria (Brice et al. J Clin Oncol. 15(3):1110-1117, 1997) to be in need of systemic therapy to treat the previously untreated FL.

The terms “Groupe d'Etude des Lymphomes Folliculaires criteria” and “GELF criteria” refer to criteria for determining whether immediate therapy for follicular lymphoma is required. In particular, a subject or individual satisfying one of more GELF criteria is determined to be in need of treatment. GELF criteria include (i) any nodal or extranodal tumor mass ≥7 cm in diameter; (ii) involvement of at least 3 nodal sites, each with diameter >3 cm; (iii) presence of type B symptoms (e.g., fever, night sweats, and weight loss); (iv) splenomegaly (>16 cm on computer tomography (CT) scan); (v) risk of local compressive symptoms that may result in organ compromise; (vi) pleural effusion or peritoneal ascites; (vii) leukemic phase (>5×10⁹/L circulating malignant cells); and (viii) cytopenia (granulocye count <1×10⁹/L and/or platelets <100×10⁹/L). See Brice et al. J Clin Oncol. 1997. In some embodiments, a subject satisfying one or more GELF criteria is considered to have a high tumor burden.

By “Follicular Lymphoma International Prognostic Index” or “FLIPI” is meant a scoring system or index for determining prognostic risk of patients (e.g., with cancer; e.g., an NHL; e.g., a follicular lymphoma (FL)). FLIPI score ranges from 0-5, depending on how many of the five conditions or risk factors a patient may have among the following: (i) age 60 years; (ii) Ann Arbor stage III-IV; hemoglobin level 120 g/L; serum lactate dehydrogenase (LDH) level upper limit of normal (ULN) (e.g., >280 units/L); and (v) number of nodal sites >4. FLIPI risk groups are defined as follows: (a) 0 or 1 FLIPI risk factors=low risk group; (b) 2 FLIPI risk factors=intermediate risk group; (c) 3-5 FLIPI risk factors=high risk group. See e.g., Solal-Céligny et al. Blood. 2004; 104 (5): 1258-1265. at Table 4.

As used herein, the terms “Ann Arbor staging” or “Ann Arbor stages” refers to a system for classification of stages of lymphoma (e.g., NHL, e.g., FL, e.g., previously untreated FL). Lymphomas (e.g., NHLs) can be classified as one of four Ann Arbor stages. Stage I refers to lymphomas exhibiting involvement of a single lymph node region or of a single extralymphatic organ or site. Stage II refers to lymphomas exhibiting involvement of 2 or more lymph node regions on the same side of the diaphragm. Stage III refers to lymphomas exhibiting involvement of lymph node regions on both sides of the diaphragm (III), which may also be accompanied by localized involvement of extralymphatic organ or site or by involvement of the spleen, or both. Stage IV refers to lymphomas exhibiting diffuse or disseminated involvement of 1 or more extralymphatic organs or tissues with or without associated lymph node enlargement. Liver involvement is always considered to be diffuse, and, thus, always considered Ann Arbor stage IV. Lymphatic structures include the lymph nodes, thymus, spleen, appendix, Waldeyer's ring, and Peyer's patches. See Carbone, P. P. et al., Cancer Res. 1971, 31(11):1860-1861.

As used herein, “treatment” (and grammatical variations thereof, such as “treat” or “treating”) refers to clinical intervention in an attempt to alter the natural course of the subject being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

As used herein, “delaying progression” of a disorder or disease means to defer, hinder, slow, retard, stabilize, and/or postpone development of the disease or disorder (e.g., a previously untreated FL). This delay can be of varying lengths of time, depending on the history of the disease and/or individual being treated. As is evident to one skilled in the art, a sufficient or significant delay can, in effect, encompass prevention, in that the individual does not develop the disease. For example, a late-stage cancer, such as development of metastasis, may be delayed.

By “extending survival” is meant increasing overall or progression free survival in a treated patient relative to an untreated patient (e.g., relative to a patient not treated with the medicament), or relative to a patient who does not express a biomarker at the designated level, and/or relative to a patient treated with an approved anti-tumor agent. An objective response refers to a measurable response, including complete response (CR) or partial response (PR).

By “reduce” or “inhibit” is meant the ability to cause an overall decrease, for example, of 20%, 30%, 40%, 50%, 60%, 70%, 75%, 80%, 85%, 90%, 95%, or greater. For clarity the term includes also reduction to zero (or below the detection limit of the analytical method), i.e., complete abolishment or elimination. In certain embodiments, reduce or inhibit can refer to the reduction or inhibition of undesirable events, such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or central nervous system (CNS) toxicities, following treatment with mosunetuzumab using the step-up dosing regimen of the invention relative to unchanging, preset dosing with the target dose of mosunetuzumab. In other embodiments, reduce or inhibit can refer to effector function of an antibody that is mediated by the antibody Fc region, such effector functions specifically including complement-dependent cytotoxicity (CDC), antibody-dependent cellular cytotoxicity (ADCC), and antibody-dependent cellular phagocytosis (ADCP). In other embodiments reduce or inhibit can refer to the symptoms of the previously untreated FL being treated, the presence or size of metastases, or the size of the primary tumor. In yet other embodiments, reducing or inhibiting cancer relapse means to reduce or inhibit tumor or cancer relapse, or tumor or cancer progression (e.g., reduce the risk of or prevent progression and/or relapse of the FL).

As used herein, “administering” is meant a method of giving a dosage of a compound (e.g., a bispecific antibody, e.g., an anti-CD20/anti-CD3 bispecific antibody, e.g., mosunetuzumab; e.g., a corticosteroid, antihistamine, anti-pyretic, interleukin-6 receptor (IL-6R) antagonist, or prophylactic agent against tumor lysis syndrome (TLS)) or a composition (e.g., a pharmaceutical composition, e.g., a pharmaceutical composition including a bispecific antibody (e.g., mosunetuzumab) or other therapeutic agent) to a subject. The compounds and/or compositions utilized in the methods described herein can be administered subcutaneously (e.g., by injection), intravenously (e.g., by intravenous infusion), or orally.

A “fixed” or “flat” dose of a therapeutic agent (e.g., a bispecific antibody) herein refers to a dose that is administered to a patient without regard for the weight or body surface area (BSA) of the patient. The fixed or flat dose is therefore not provided as a mg/kg dose or a mg/m 2 dose, but rather as an absolute amount of the therapeutic agent (e.g., mg).

A “subject” or an “individual” is a mammal. Mammals include, but are not limited to, primates (e.g., humans and non-human primates such as monkeys), domesticated animals (e.g., cows, sheep, cats, dogs, and horses), rabbits, and rodents (e.g., mice and rats). In a particular embodiment, the subject or individual is a human.

“Individual response” or “response” can be assessed using any endpoint indicating a benefit to the subject, including, without limitation, (1) inhibition, to some extent, of disease progression (e.g., progression of a previously untreated FL, including slowing down and complete arrest; (2) a reduction in tumor size; (3) inhibition (i.e., reduction, slowing down or complete stopping) of cancer cell infiltration into adjacent peripheral organs and/or tissues; (4) inhibition (i.e., reduction, slowing down or complete stopping) of metastasis; (5) relief, to some extent, of one or more symptoms associated with the previously untreated FL; (6) increase or extend in the length of survival, including overall survival and progression-free survival; and/or (9) decreased mortality at a given point of time following treatment. In some embodiments, response to treatment of a previously untreated FL is evaluated using the Lugano response criteria for malignant lymphoma (Cheson B D, et al. J Clin Oncol 2014; 32:1-9).

The “Lugano Criteria” or “Lugano Response Criteria” for malignant lymphoma, as used herein, are a set of criteria for evaluating response assessments (e.g., of PET/CT scans) to the methods of treatment described herein. The Lugano Criteria is described below in Table 1:

TABLE 1
Lugano Response Criteria for Malignant Lymphoma Response Assessment

| Response and | | |
|---|---|---|
| Site | PET/CT-Based Response | CT-Based Response |
| | | Complete radiologic response (all |
| Complete | Complete metabolic response | of the following) |
| Lymph nodes and | Score 1, 2, or 3^{a} with or without a | Target nodes/nodal masses must |
| extralymphatic sites | residual mass on 5PS^{b} | regress to ≤1.5 cm in LDi |
| | It is recognized that in Waldeyer ring | No extralymphatic sites of disease |
| | or extranodal sites with high | |
| | physiologic uptake or with activation | |
| | within spleen or marrow (e.g., with | |
| | chemotherapy or myeloid colony- | |
| | stimulating factors), uptake may be | |
| | greater than normal mediastinum | |
| | and/or liver. In this circumstance, | |
| | complete metabolic response may be | |
| | inferred if uptake at sites of initial | |
| | involvement is no greater than | |
| | surrounding normal tissue even if the | |
| | tissue has high physiologic uptake. | |
| Non-measured | Not applicable | Absent |
| lesion | | |
| Organ enlargement | Not applicable | Regress to normal |
| New lesions | None | None |
| Bone marrow | No evidence of FDG-avid disease in | Normal by morphology; if |
| | marrow | indeterminate, IHC negative |
| | | Partial remission (all of the |
| Partial response | Partial metabolic response | following) |
| Lymph nodes and | Score 4 or 5^{b} with reduced uptake | ≥50% decrease in SPD of up to 6 |
| extralymphatic sites | compared with baseline and residual | target measurable nodes and |
| | mass(es) of any size | extranodal sites |
| | At interim, these findings suggest | When a lesion is too small to measure |
| | responding disease | on CT, assign 5 × 5 mm as the |
| | At interim, these findings suggest | default value |
| | responding disease | When no longer visible, 0 × 0 mm |
| | | For a node > 5 × 5 mm but smaller |
| | | than normal, use actual measurement |
| | | for calculation |
| Non-measured | | Absent or normal, regressed, but no |
| lesion | Not applicable | increase |
| Organ enlargement | Not applicable | Spleen must have regressed by |
| | | >50% in length beyond normal |
| New lesions | None | None |
| Bone marrow | Residual uptake higher than uptake in | Not applicable |
| | normal marrow but reduced | |
| | compared with baseline (diffuse | |
| | uptake compatible with reactive | |
| | changes from chemotherapy | |
| | allowed). If there are persistent focal | |
| | changes in the marrow in the context | |
| | of a nodal response, consideration | |
| | should be given to further evaluation | |
| | with MRI or biopsy or an interval | |
| | scan. | |
| No response or | | |
| stable disease | No metabolic response | Stable disease |
| Target nodes/nodal | Score 4 or 5^{b} with no significant | <50% decrease from baseline in |
| masses, extranodal | change in FDG uptake from baseline | SPD of up to 6 dominant, measurable |
| lesions | at interim or end of treatment | nodes and extranodal sites; no criteria |
| | | for progressive disease are met |
| Non-measured | Not applicable | No increase consistent with |
| lesion | | progression |
| Organ enlargement | Not applicable | No increase consistent with |
| | | progression |
| New lesions | None | None |
| Bone marrow | No change from baseline | Not applicable |
| Progressive | | Progressive disease requires at |
| disease | Progressive metabolic disease | least one of the following: |
| Individual target | Score 4 or 5^{b} with an increase in | PPD progression |
| nodes/nodal | intensity of uptake from baseline | |
| masses | and/or | |
| Extranodal lesions | New FDG-avid foci consistent with | An individual node/lesion must be |
| | lymphoma at interim or end-of- | abnormal with: LDi > 1.5 cm and |
| | treatment assessment | Increase by ≥50% from PPD nadir |
| | | and An increase in LDi or SDi from |
| | | nadir 0.5 cm for lesions ≤ 2 cm 1.0 cm |
| | | for lesions >2 cm In the setting of |
| | | splenomegaly (>13 cm), the splenic |
| | | length must increase by >50% of the |
| | | extent of its prior increase beyond |
| | | baseline (e.g., a 15 cm spleen must |
| | | increase to >16 cm). If no prior |
| | | splenomegaly, must increase by at |
| | | least 2 cm from baseline. |
| | | New or recurrent splenomegaly. |
| | | New or clear progression of |
| | | preexisting non-measured lesions. |
| New lesions | New FDG-avid foci consistent with | Regrowth of previously resolved |
| | lymphoma rather than another | lesions. |
| | etiology (e.g., infection, inflammation); | A new node >1.5 cm in any axis |
| | if uncertain regarding etiology of new | A new extranodal site >1.0 cm in any |
| | lesions, biopsy or interval scan may | axis; if <1.0 cm in any axis, its |
| | be considered | presence must be unequivocal and |
| | | must be attributable to lymphoma. |
| | | Assessable disease of any size |
| | | unequivocally attributable to |
| | | lymphoma. |
| Bone marrow | New or recurrent FDG-avid foci | New or recurrent involvement |

5PS = 5-point scale;
CT = computed tomography;
FDG = fluorodeoxyglucose;
IHC = immunohistochemistry;
LDi = longest transverse diameter of a lesion;
MRI = magnetic resonance imaging;
PET = positron emission tomography;
PPD = cross product of the LDi and perpendicular diameter;
SDi = shortest axis perpendicular to the LDi;
SPD = sum of the product of the perpendicular diameters for multiple lesions.
^{a}A score of 3 in many patients indicates a good prognosis with standard treatment, especially if at the time of an interim scan.
However, in study involving PET in which de-escalation is investigated, it may be preferable to consider a score of 3 as an inadequate response (to avoid under treatment).
Measured dominant lesions: Up to six of the largest dominant nodes, nodal masses, and extranodal lesions selected to be clearly measurable in two diameters.
Nodes should preferably be from disparate regions of the body and should include, where applicable, mediastinal and retroperitoneal areas.
Non-nodal lesions include those in solid organs (e.g., liver, spleen, kidneys, lungs), gastrointestinal involvement, cutaneous lesions, or those noted on palpation.
Non-measured lesions: Any disease not selected as measured; dominant disease and truly assessable disease should be considered not measured.
These sites include any nodes, nodal masses, and extranodal sites not selected as dominant or measurable or that do not meet the requirements for measurability but are still considered abnormal, as well as truly assessable disease, which is any site of suspected disease that would be difficult to follow quantitatively with measurement, including pleural effusions, ascites, bone lesions, leptomeningeal disease, abdominal masses, and other lesions that cannot be confirmed and followed by imaging.
In Waldeyer's ring or in extranodal sites (e.g., gastrointestinal tract, liver, bone marrow), FDG uptake may be greater than in the mediastinum with complete metabolic response, but should be no higher than surrounding normal physiologic uptake (e.g., with marrow activation as a result of chemotherapy or myeloid growth factors).
^{b}PET 5PS: 1 = no uptake above background;
2 = uptake ≤ mediastinum;
3 = uptake > mediastinum but > liver;
4 = uptake moderately > liver;
5 = uptake markedly higher than liver and/or new lesions;
X = new areas of uptake unlikely to be related to lymphoma.
See Cheson BD, et al. J Clin Oncol 2014; 32:1-9.

For purposes of evaluation, target lesions include up to six of the largest target nodes, nodal masses, or other lymphomatous lesions that are measurable in two diameters should be identified from different body regions representative of the patient's overall disease burden and include mediastinal and retroperitoneal disease, if involved. At baseline, a measurable node must be greater than 15 mm in longest diameter (LDi). Measurable extranodal disease may be included in the six representative, measured lesions. At baseline, measurable extranodal lesions should be greater than 10 mm LDi. All other lesions (including nodal, extranodal, and assessable disease) should be followed as non-measured disease as non-target lesions (e.g., cutaneous, gastrointestinal, bone, spleen, liver, kidneys, pleural or pericardial effusions, ascites, bone, bone marrow). Lesions may split or may become confluent over time. In the case of split lesions, the individual product of the perpendicular diameters (PPDs) of the nodes should be summed together to represent the PPD of the split lesion; this PPD is added to the sum of the PPDs of the remaining lesions to measure response. If subsequent growth of any or all of these discrete nodes occurs, the nadir of each individual node is used to determine progression. In the case of confluent lesions, the PPD of the confluent mass should be compared with the sum of the PPDs of the individual nodes, with more than 50% increase in PPD of the confluent mass compared with the sum of individual nodes necessary to indicate progressive disease. The LDi and smallest diameter (SDi) are no longer needed to determine progression.

As used herein, “objective response rate” (ORR) refers to the sum of complete response (CR) rate and partial response (PR) rate.

As used herein, “duration of objective response” (DOR) is defined as the time from the first occurrence of a documented objective response to disease progression, relapse, or death from any cause, whichever occurs first.

As used herein, “duration of complete response” (DOCR) is defined as the time from the first occurrence of a documented complete response to disease progression, relapse, or death from any cause, whichever occurs first.

As used herein, “tumor burden” refers to the total amount of tumor (e.g., tumor cells or tumor mass) in a subject (e.g., a human subject) having a cancer, e.g., an NHL, e.g., an FL. In some embodiments, tumor burden is defined as the sum of diameters of target lesions or the sum of the product of target lesions. In a particular embodiment, tumor burden is defined as the sum of the product of the diameters of (SPD) target lesions. In some embodiments, the diameters of target lesions is quantified by computed tomography (CT).

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

An “effective response” of a subject or a subject's “responsiveness” to treatment with a medicament and similar wording refers to the clinical or therapeutic benefit imparted to a subject as risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A subject who “does not have an effective response” to treatment refers to a subject who does not have any one of extending survival (including overall survival and progression free survival); resulting in an objective response (including a complete response or a partial response); or improving signs or symptoms of cancer.

As used herein, “survival” refers to the patient remaining alive, and includes overall survival as well as progression-free survival.

As used herein, “overall survival” (OS) refers to the percentage of subjects in a group who are alive after a particular duration of time, e.g., 1 year or 5 years from the time of diagnosis or treatment.

As used herein, “progression-free survival” (PFS) refers to the length of time during and after treatment during which the disease being treated (e.g., a previously untreated FL) does not get worse. Progression-free survival may include the amount of time patients have experienced a complete response or a partial response, as well as the amount of time patients have experienced stable disease.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

The terms “full-length antibody,” “intact antibody,” and “whole antibody” are used herein interchangeably to refer to an antibody having a structure substantially similar to a native antibody structure or having heavy chains that contain an Fc region as defined herein.

The term “cluster of differentiation 3” or “CD3,” as used herein, refers to any native CD3 from any vertebrate source, including mammals such as primates (e.g., humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD3γ protein (NCBI RefSeq No. NP_000064), which is 182 amino acids in length.

The term “cluster of differentiation 20” or “CD20,” as used herein, refers to any native CD20 from any vertebrate source, including mammals such as primates (e.g., humans) and rodents (e.g., mice and rats), unless otherwise indicated. The term encompasses “full-length,” unprocessed CD20, as well as any form of CD20 that results from processing in the cell. The term also encompasses naturally occurring variants of CD20, including, for example, splice variants or allelic variants. CD20 includes, for example, human CD20 protein (see, e.g., NCBI RefSeq Nos. NP_068769.2 and NP_690605.1), which is 297 amino acids in length and may be generated, for example, from variant mRNA transcripts that lack a portion of the 5′ UTR (see, e.g., NCBI RefSeq No. NM_021950.3) or longer variant mRNA transcripts (see, e.g., NCBI RefSeq No. NM_152866.2).

The terms “anti-CD20/anti-CD3 bispecific antibody,” “bispecific anti-CD20/anti-CD3 antibody,” and “antibody that binds to CD20 and CD3,” or variants thereof, refer to mosunetuzumab.

As used herein, the term “mosunetuzumab” refers to an anti-CD20/anti-CD3 bispecific antibody having the International Nonproprietary Names for Pharmaceutical Substances (INN) List 117 (WHO Drug Information, Vol. 31, No. 2, 2017, p. 304-305), or the CAS Registry Number 1905409-39-3.

As used herein, the term “lenalidomide” refers to a compound having the CAS Registry Number 191732-72-6 and IUPAC name (3RS)-3-(4-Amino-1-oxo-1,3-dihydro-2H-isoindol-2-yl)piperidine-2,6-dione. Lenalidomide is also known by tradenames including REVLIMID®, linamid, and lenalid. Lenalidomide has the DrugBank Accession Number DB00480, PubChem CID 216326, and chemical formula C₁₃H₁₃N₃O₃.

As used herein, the term “binds,” “specifically binds to,” or is “specific for” refers to measurable and reproducible interactions such as binding between a target and an antibody, which is determinative of the presence of the target in the presence of a heterogeneous population of molecules including biological molecules. For example, an antibody that specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, for example, by a radioimmunoassay (RIA). In certain embodiments, an antibody that specifically binds to a target has a dissociation constant (K_{D}) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding. The term as used herein can be exhibited, for example, by a molecule having a K_{D} for the target of 10⁻⁴ M or lower, alternatively 10⁻⁵ M or lower, alternatively 10⁻⁶ M or lower, alternatively 10⁻⁷ M or lower, alternatively 10⁻⁸ M or lower, alternatively 10⁻⁸ M or lower, alternatively 10⁻¹⁸ M or lower, alternatively 10⁻¹¹ M or lower, alternatively 10⁻¹² M or lower or a K_{D} in the range of 10⁻⁴ M to 10⁻⁶ M or 10⁻⁶ M to 10⁻¹⁰ M or 10⁻⁷ M to 10⁻⁹ M. As will be appreciated by the skilled artisan, affinity and K_{D} values are inversely related. A high affinity for an antigen is measured by a low K_{D} value. In one embodiment, the term “specific binding” refers to binding where a molecule binds to a particular polypeptide or epitope on a particular polypeptide without substantially binding to any other polypeptide or polypeptide epitope.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, the term “chemotherapeutic agent” refers to a compound useful in the treatment of a cancer, such as a previously untreated FL. Examples of chemotherapeutic agents include EGFR inhibitors (including small molecule inhibitors (e.g., erlotinib (TARCEVA®, Genentech/OSI Pharm.); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); and dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine)); a tyrosine kinase inhibitor (e.g., an EGFR inhibitor; a small molecule HER2 tyrosine kinase inhibitor such as TAK165 (Takeda); CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; PKI-166 (Novartis); pan-HER inhibitors such as canertinib (CI-1033; Pharmacia); Raf-1 inhibitors such as antisense agent ISIS-5132 (ISIS Pharmaceuticals) which inhibit Raf-1 signaling; non-HER-targeted tyrosine kinase inhibitors such as imatinib mesylate (GLEEVEC®, Glaxo SmithKline); multi-targeted tyrosine kinase inhibitors such as sunitinib (SUTENT®, Pfizer); VEGF receptor tyrosine kinase inhibitors such as vatalanib (PTK787/ZK222584, Novartis/Schering AG); MAPK extracellular regulated kinase I inhibitor CI-1040 (Pharmacia); quinazolines, such as PD 153035, 4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g., those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone); and rapamycin (sirolimus, RAPAMUNE®)); proteasome inhibitors such as bortezomib (VELCADE®, Millennium Pharm.); disulfiram; epigallocatechin gallate; salinosporamide A; carfilzomib; 17-AAG (geldanamycin); radicicol; lactate dehydrogenase A (LDH-A); fulvestrant (FASLODEX®, AstraZeneca); letrozole (FEMARA®, Novartis), finasunate (VATALANIB®, Novartis); oxaliplatin (ELOXATIN®, Sanofi); 5-FU (5-fluorouracil); leucovorin; lonafamib (SCH 66336); sorafenib (NEXAVAR®, Bayer Labs); AG1478, alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin γ1 and calicheamicin ω1); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, cactinomycin, carabicin, caminomycin, carzinophilin, chromomycinis, dactinomycin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); thiotepa; chloranmbucil; GEMZAR® (gemcitabine); 6-thioguanine; mercaptopurine; methotrexate; etoposide (VP-16); ifosfamide; mitoxantrone; novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids, prodrugs, and derivatives of any of the above.

Chemotherapeutic agents also include (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; (ix) growth inhibitory agents including vincas (e.g., vincristine and vinblastine), NAVELBINE® (vinorelbine), taxanes (e.g., paclitaxel, nab-paclitaxel, and docetaxel), topoisomerase II inhibitors (e.g., doxorubicin, epirubicin, daunorubicin, etoposide, and bleomycin), and DNA alkylating agents (e.g., tamoxigen, dacarbazine, mechlorethamine, cisplatin, methotrexate, 5-fluorouracil, and ara-C); and (x) pharmaceutically acceptable salts, acids, prodrugs, and derivatives of any of the above.

The term “chemo-immunotherapy” refers to combination therapy that includes both chemotherapy drugs and immunotherapeutic agents. In some embodiments, chemo-immunotherapy is used to treat a cancer, e.g., a CD20-positive cancer, e.g., a NHL, e.g., a FL. In some embodiments, immunotherapeutic agents include an antibody, e.g., an anti-CD20 antibody (e.g., an anti-CD20 monoclonal antibody). In some embodiments, the anti-CD20 antibody or anti-CD20 monoclonal antibody is rituximab or obinutuzumab. In some embodiments, chemo-immunotherapy includes R-CHOP.

The term “cytotoxic agent” as used herein refers to any agent that is detrimental to cells (e.g., causes cell death, inhibits proliferation, or otherwise hinders a cellular function). Cytotoxic agents include, but are not limited to, radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb, and radioactive isotopes of Lu); chemotherapeutic agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signaling inhibitors, HDAC inhibitors, proteasome inhibitors, and inhibitors of cancer metabolism. In one instance, the cytotoxic agent is a platinum-based chemotherapeutic agent (e.g., carboplatin or cisplatin). In one instance, the cytotoxic agent is an antagonist of EGFR, e.g., N-(3-ethynylphenyl)-6,7-bis(2-methoxyethoxy)quinazolin-4-amine (e.g., erlotinib). In one instance the cytotoxic agent is a RAF inhibitor, e.g., a BRAF and/or CRAF inhibitor. In one instance the RAF inhibitor is vemurafenib. In one instance, the cytotoxic agent is a PI3K inhibitor.

The term “package insert” is used to refer to instructions customarily included in commercial packages of therapeutic products, that contain information about the indications, usage, dosage, administration, combination therapy, contraindications and/or warnings concerning the use of such therapeutic products.

### III. Therapeutic Methods

Provided herein are methods of treating a subject having a previously untreated follicular lymphoma (FL) by administration of mosunetuzumab and lenalidomide as a combination therapy. In particular, the present invention relates to methods of treating a subject having a 1/L FL by subcutaneous administration of mosunetuzumab and oral administration of lenalidomide. In some embodiments, the FL is a Graded FL (e.g., Grade 1, 2, or 3a, but not Grade 3b FL). In some embodiments, the FL of each subject is histologically documented as Grade 1, 2, or 3a, but not 3b, according to the World Health Organization classification of lymphoid neoplasms (as referenced in Swerdlow S H, et al. Blood 2016; 127:2375-90).

In some instances, the subject has not been treated with any standard or investigational anti-cancer therapy, including, but not limited to: (i) lenalidomide exposure within 12 months prior to administration of the first dose of mosunetuzumab (e.g., C1D1 dose) of the methods of the invention; (ii) fludarabine or alemtuzumab within 12 months prior to administration of the first dose of mosunetuzumab (e.g., C1D1 dose) of the methods of the invention; (iii) radioimmunoconjugate within 12 weeks prior to administration of the first dose of mosunetuzumab (e.g., C1D1 dose) of the methods of the invention; (iv) prior anti-lymphoma treatment with monoclonal antibody or antibody-drug conjugate within 4 weeks prior to administration of the first dose of mosunetuzumab (e.g., C1D1 dose) of the methods of the invention; and (v) treatment with any chemotherapeutic agent, or treatment with any other anti-cancer agent (investigational or otherwise) within 4 weeks or 5 half-lives of the drug, whichever is shorter, prior to administration of the first dose of mosunetuzumab (e.g., C1D1 dose) of the methods of the invention.

In some embodiments, the subject has been previously determined based on the Groupe d'Etude des Lymphomes Folliculaires (G ELF) criteria (Brice et al. J Clin Oncol. 15(3):1110-1117, 1997) to be in need of systemic therapy to treat the previously untreated FL.

### A. Therapeutic Methods for Dosing of Mosunetuzumab and Lenalidomide

The present invention relates to methods of treating a subject having a previously untreated follicular lymphoma (FL) by administration of mosunetuzumab and lenalidomide as a combination therapy. In particular, the present invention relates to methods of treating a subject having a previously untreated FL by subcutaneous administration of mosunetuzumab and oral administration of lenalidomide.

In one aspect, the invention features a method of treating a subject having a previously untreated follicular lymphoma (FL), the method comprising administering to the subject mosunetuzumab and lenalidomide according to a dosing regimen comprising a first 21-day (±1 day) dosing cycle and a second 28-day (±1 day) dosing cycle, wherein: (a) the first dosing cycle comprises a first dose (C1D1), a second dose (C1D2), and a third dose (C1D3) of mosunetuzumab subcutaneously administered on Days 1, 8 (±1 day), and 15 (±1 day), respectively, of the first dosing cycle, wherein the C1D1 is about 5 mg (e.g., 5 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, or ±1 mg; e.g., 5 mg), the C1D2 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and the C1D3 is about mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), (b) the second dosing cycle comprises a single dose (C2D1) of mosunetuzumab subcutaneously administered on Day 1 of the second dosing cycle, wherein the C2D1 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and (c) the second dosing cycle further comprises orally administering between about 5 mg and about 20 mg (e.g., between about 5 mg and about 10 mg, between about mg and about 15 mg, between about 15 mg and about 20 mg, or between about 5 mg and about mg; e.g., about 5, 6, 7, 8, 9, 10, 11, 12, 13, 14, 15, 16, 17, 18, 19, or 20 mg) lenalidomide daily on Days 1-21 of the second dosing cycle. In some embodiments, the first dosing cycle is a three-dose dosing cycle (i.e., comprising three doses of mosunetuzumab).

In some embodiments, the dosing regimen comprises one or more (e.g., 1, 2, 3, 4, 5, 6, 7, 8, 9, 10, or more) additional dosing cycles. In some embodiments, the dosing regimen comprises one to ten (e.g., 1, 2, 3, 4, 5, 6, 7, 8, 9, or 10) additional dosing cycles. In some embodiments, the dosing regimen comprises ten additional dosing cycles. In some embodiments, the length of any of the one or more additional dosing cycles is about 28 days (±1 day). In some embodiments, the length of each of the one or more additional dosing cycles is about 28 days (±1 day).

In some embodiments, any of the one or more additional dosing cycles comprises an additional single dose of mosunetuzumab. In some embodiments, each of the one or more additional dosing cycles comprises an additional single dose of mosunetuzumab. In some embodiments, the method comprises subcutaneously administering to the subject each additional single dose of mosunetuzumab on Day 1 of each of the one or more additional dosing cycles. In some embodiments, the additional single dose of mosunetuzumab is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg). In some embodiments, each additional single dose of mosunetuzumab is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg).

In some embodiments, lenalidomide is not administered during the first dosing cycle. In some embodiments, lenalidomide is orally administered during any of the one or more (e.g., 1, 2, 3, 4, 5, 6, 7, 8, 9, 10, or more) additional dosing cycles. In some embodiments, lenalidomide is orally administered during any of the ten additional dosing cycles. In some embodiments, lenalidomide is orally administered during each of the ten additional dosing cycles. In some embodiments, lenalidomide is orally administered on Days 1-21 of each of the additional dosing cycles comprising administration of lenalidomide. In some embodiments, lenalidomide is not administered on the last 7 days (±1 day) of any dosing cycle comprising administration of lenalidomide. In some embodiments, lenalidomide is administered at a dose of about 10 mg (e.g., 10 mg±0.025 mg, ±0.05 mg, ±0.075 mg, ±0.1 mg, ±0.2 mg, ±0.3 mg, ±0.4 mg, ±0.5 mg, ±0.75 mg, ±1 mg, or ±2 mg; e.g., 10 mg). In some embodiments, lenalidomide is administered at a dose of about 20 mg (e.g., 20 mg±0.05 mg, ±0.1 mg, ±0.2 mg, ±0.3 mg, ±0.4 mg, ±0.5 mg, ±0.75 mg, ±1 mg, ±1.5 mg, ±2 mg, ±3 mg, or ±4 mg; e.g., 20 mg).

In one aspect, the invention features a method of treating a subject having a previously untreated FL, the method comprising administering to the subject mosunetuzumab and lenalidomide according to a dosing regimen comprising a first 21-day (±1 day) dosing cycle and a second 28-day (±1 day) dosing cycle, wherein: (a) the first dosing cycle comprises a first dose (C1D1), a second dose (C1D2), and a third dose (C1D3) of mosunetuzumab subcutaneously administered on Days 1, 8 (±1 day), and 15 (±1 day), respectively, of the first dosing cycle, wherein the C1D1 is about 5 mg (e.g., 5 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, or ±1 mg; e.g., 5 mg), the C1D2 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and the C1D3 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), (b) the second dosing cycle comprises a single dose (C2D1) of mosunetuzumab subcutaneously administered on Day 1 of the second dosing cycle, wherein the C2D1 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and (c) the second dosing cycle further comprises orally administering about 10 mg (e.g., 10 mg±0.025 mg, ±0.05 mg, ±0.075 mg, ±0.1 mg, ±0.2 mg, ±0.3 mg, ±0.4 mg, ±0.5 mg, ±0.75 mg, ±1 mg, or ±2 mg; e.g., 10 mg) lenalidomide daily on Days 1-21 of the second dosing cycle.

In one aspect, the invention features a method of treating a subject having a previously untreated FL, the method comprising administering to the subject mosunetuzumab and lenalidomide according to a dosing regimen comprising a first 21-day (±1 day) dosing cycle and a second 28-day (±1 day) dosing cycle, wherein: (a) the first dosing cycle comprises a first dose (C1D1), a second dose (C1D2), and a third dose (C1D3) of mosunetuzumab subcutaneously administered on Days 1, 8 (±1 day), and 15 (±1 day), respectively, of the first dosing cycle, wherein the C1D1 is about 5 mg (e.g., 5 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, or ±1 mg; e.g., 5 mg), the C1D2 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and the C1D3 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), (b) the second dosing cycle comprises a single dose (C2D1) of mosunetuzumab subcutaneously administered on Day 1 of the second dosing cycle, wherein the C2D1 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and (c) the second dosing cycle further comprises orally administering about 20 mg (e.g., 20 mg±0.05 mg, ±0.1 mg, ±0.2 mg, ±0.3 mg, ±0.4 mg, ±0.5 mg, ±0.75 mg, ±1 mg, ±1.5 mg, ±2 mg, ±3 mg, or ±4 mg; e.g., 20 mg) lenalidomide daily on Days 1-21 of the second dosing cycle.

In one aspect, the invention features a method of treating a subject having a previously untreated FL, the method comprising administering to the subject mosunetuzumab and lenalidomide according to a dosing regimen comprising a first 21-day (±1 day) dosing cycle and eleven subsequent 28-day (±1 day) dosing cycles, wherein: (a) the first dosing cycle comprises a first dose (C1D1), a second dose (C1D2), and a third dose (C1D3) of mosunetuzumab subcutaneously administered on Days 1, 8 (±1 day), and 15 (±1 day), respectively, of the first dosing cycle, wherein the C1D1 is about 5 mg (e.g., 5 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, or ±1 mg; e.g., 5 mg), the C1D2 is about 45 mg (e.g., 45 mg 0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and the C1D3 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), (b) the second to twelfth dosing cycles each comprises a single dose (C2D1-C12D1) of mosunetuzumab subcutaneously administered on Day 1 of each dosing cycle, wherein each single dose C2D1-C12D1 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and (c) the second to twelfth dosing cycles each further comprises orally administering about 10 mg (e.g., 10 mg±0.025 mg, ±0.05 mg, ±0.075 mg, ±0.1 mg, ±0.2 mg, ±0.3 mg, ±0.4 mg, ±0.5 mg, ±0.75 mg, ±1 mg, or ±2 mg; e.g., 10 mg) lenalidomide daily on Days 1-21 of each dosing cycle.

In one aspect, the invention features a method of treating a subject having a previously untreated FL, the method comprising administering to the subject mosunetuzumab and lenalidomide according to a dosing regimen comprising a first 21-day (±1 day) dosing cycle and eleven subsequent 28-day (±1 day) dosing cycles, wherein: (a) the first dosing cycle comprises a first dose (C1D1), a second dose (C1D2), and a third dose (C1D3) of mosunetuzumab subcutaneously administered on Days 1, 8 (±1 day), and 15 (±1 day), respectively, of the first dosing cycle, wherein the C1D1 is about 5 mg (e.g., 5 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, or ±1 mg; e.g., 5 mg), the C1D2 is about 45 mg (e.g., 45 mg 0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and the C1D3 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), (b) the second to twelfth dosing cycles each comprises a single dose (C2D1-C12D1) of mosunetuzumab subcutaneously administered on Day 1 of each dosing cycle, wherein each single dose C2D1-C12D1 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and (c) the second to twelfth dosing cycles each further comprises orally administering about 20 mg (e.g., 20 mg±0.05 mg, ±0.1 mg, ±0.2 mg, ±0.3 mg, ±0.4 mg, ±0.5 mg, ±0.75 mg, ±1 mg, ±1.5 mg, ±2 mg, ±3 mg, or ±4 mg; e.g., 20 mg) lenalidomide daily on Days 1-21 of each dosing cycle.

In some embodiments, the FL is histologically documented as Grade 1, 2, or 3a, but not 3b according to the World Health Organization classification of lymphoid neoplasms (as referenced in Swerdlow S H, et al. Blood 2016; 127:2375-90).

In some embodiments, the subject has been previously determined based on the Groupe d'Etude des Lymphomes Folliculaires (GELF) criteria (Brice et al. J Clin Oncol. 15(3):1110-1117, 1997) to be in need of systemic therapy to treat the previously untreated FL.

In some embodiments, the subject is human.

For all the methods described herein, mosunetuzumab and lenalidomide are formulated, dosed, and administered in a fashion consistent with good medical practice. Factors for consideration in this context include the particular disorder being treated, the particular mammal being treated, the clinical condition of the individual subject, the cause of the disorder, the site of delivery of the agent, the method of administration, the scheduling of administration, and other factors known to medical practitioners. Mosunetuzumab and lenalidomide need not be, but are optionally formulated with, administered in combination with, or used with one or more additional agents (e.g., therapeutic agents) currently used to prevent or treat the disorder in question and/or reduce the rate of, reduce the severity of, treat, or prevent adverse events and/or symptoms associated with the adverse events. The effective amount of such other agents depends on the amount of mosunetuzumab and/or lenalidomide present in the formulation, the type of disorder or treatment, the type of adverse event, and other factors discussed herein. Mosunetuzumab and lenalidomide may be suitably administered to the subject over a series of treatments. When mosunetuzumab and lenalidomide are administered on the same day, lenalidomide is administered to the subject prior to administration of mosunetuzumab.

### B. Dosing Strategies for Mitigating Adverse Events

The present invention relates to methods of treating a subject having a previously untreated follicular lymphoma (FL) by administration of mosunetuzumab and lenalidomide as a combination therapy. In some embodiments, therapies and dosing regimens described herein provide acceptable safety profiles in subjects with previously untreated FL treated with the described dosing regimens.

1. CRS Symptoms and Grading

Any of the methods described herein may involve monitoring a subject for cytokine release syndrome (CRS), e.g., a CRS event following commencement of any of the methods described above. Current clinical management focuses on treating the individual signs and symptoms, providing supportive care, and attempting to dampen the inflammatory response using a high dose of corticosteroids. However, this approach is not always successful, especially in the case of late intervention. The CRS grading criteria used by the methods described herein are published by the American Society for Transplantation and Cellular Therapy (ASTCT) to define mild, moderate, severe, or life-threatening CRS and harmonize reporting across clinical trials to allow rapid recognition and treatment of CRS (Lee et al. Biol Blood Marrow Transplantation. 25(4): 625-638, 2019). The ASTCT criteria is intended to be objective, easy to apply, and more accurately categorize the severity of CRS. This CRS grading system is shown below in Table 2.

TABLE 2
CRS Grading System

| CRS | | | | |
|---|---|---|---|---|
| Parameter | Grade 1 | Grade 2 | Grade 3 | Grade 4 |
| Fever | Temperature | Temperature | Temperature | Temperature |
| | ≥38° C. | ≥38° C. | ≥38° C. | ≥38° C. |
| | | with | | |
| Hypotension | None | Not requiring | Requiring a | Requiring multiple |
| | | vasopressors | vasopressor with or | vasopressors |
| | | | without vasopressin | (excluding |
| | | | | vasopressin) |
| | | and/or | | |
| Hypoxia | None | Requiring low-glow | Requiring high-flow | Requiring positive |
| | | nasal cannula or | nasal cannula, | pressure |
| | | blow-by | facemask, | (e.g., CPAP, BiPAP, |
| | | | nonrebreather mask | intubation and |
| | | | or Venturi mask | mechanical |
| | | | | ventilation) |

ASTCT = American Society for Transplantation and Cellular Therapy;
BiPAP = bilevel positive airway pressure;
CPAP = continuous positive airway pressure;
CRS = cytokine release syndrome;
CTCAE = Common Terminology Criteria for Adverse Events.

Fever is defined as a temperature 38° C. not attributable to any other cause. In subjects who have CRS then receive antipyretic or anticytokine therapy such as tocilizumab or corticosteroids, fever is no longer required to grade subsequent CRS severity. In this case, CRS grading is determined by hypotension and/or hypoxia.

CRS grade is determined by the more severe event, hypotension or hypoxia not attributable to any other cause. For example, a subject with temperature of 39.5° C., hypotension requiring 1 vasopressor, and hypoxia requiring low-flow nasal cannula is classified as Grade 3 CRS.

Low-flow nasal cannula is defined as oxygen delivered at 6 L/minute. Low flow also includes blow-by oxygen delivery, sometimes used in pediatrics. High-flow nasal cannula is defined as oxygen delivered at >6 L/minute.

CRS is associated with elevations in a wide array of cytokines, including marked elevations in IFN-γ, IL-6, and TNF-α levels. Emerging evidence implicates IL-6, in particular, as a central mediator in CRS. IL-6 is a proinflammatory, multi-functional cytokine produced by a variety of cell types, which has been shown to be involved in a diverse array of physiological processes, including T cell activation. Regardless of the inciting agent, CRS is associated with high IL-6 levels (Nagorsen et al. Cytokine. 25(1): 31-5, 2004; Lee et al. Blood. 124(2): 188-95, 2014); Doesegger et al. Clin. Transl. Immunology. 4(7): e39, 2015), and IL-6 correlates with the severity of CRS, with subjects who experience a Grade 4 or 5 CRS event having much higher IL-6 levels compared to subjects who do not experience CRS or experience milder CRS (Grades 0-3) (Chen et al. J. Immunol. Methods. 434:1-8, 2016).

Therefore, blocking the inflammatory action of IL-6 using an agent that inhibits IL-6-mediated signaling to manage CRS observed in subjects during the double-step fractionated, dose-escalation dosing regimen is an alternative to steroid treatment that would not be expected to negatively impact T cell function or diminish the efficacy or clinical benefit of mosunetuzurriab therapy in the treatment of CD20-positive cell proliferative disorders (e.g., a B cell proliferative disorders).

If the subject has a CRS event that does not resolve or worsens within 24 hours of administering the IL-6R antagonist to treat the symptoms of the CRS event, and the method may further comprise administering to the subject one or more additional doses of the IL-6R antagonist to manage the CRS event. The subject may be administered a corticosteroid, such as methylprednisolone or dexamethasone if CRS event is not managed through administration of the IL-6R antagonist.

2. Other Adverse Events and Grading

Any of the methods described herein may involve monitoring a subject for additional non-CRS adverse events. Incidence, nature, and severity of physical findings and adverse events, with severity determined according to the National Cancer Institute Common Terminology Criteria for Adverse Events version 5 (NCI CTCAE v5.0). Other than CRS, one of the most common adverse events reported in patients undergoing treatment with mosunetuzumab and/or lenalidomide is neutropenia (e.g., febrile neutropenia).

Neutropenia is characterized by an abnormally low blood count of neutrophils, which are a type of white blood cells. Neutropenia may lead to an increased risk of infection. The generally accepted reference range for absolute neutrophil count (ANC) in adult humans is 1,500 to 8,000 cells/μL of blood. Mild neutropenia is characterized by ANC between 1,000-1,500 cells/μL (Grade 1-2); moderate neutropenia is characterized by ANC between 500 and 1,000 cells/μL (Grade 3), and severe neutropenia is characterized by ANC below 500 cells/μL (Grade 4). Febrile neutropenia (Grade 3+ neutropenia) is characterized by ANC below 1,000 cells/μL in addition to either a single temperature measurement greater than 38.3° C. or sustained temperature measurements greater than 38° C. for more than one hour.

Tumor lysis syndrome (TLS) is a risk of anti-tumor therapy in hematologic malignancies, including NHL. Metabolic abnormalities associated with TLS (Howard Criteria; Howard et al. N. Engl. J. Med. 364(19)1844-1854, 2011) are described in Table 3 below:

TABLE 3
Metabolic Abnormalities associated with Laboratory and Clinical Tumor Lysis Syndrome

| Metabolic | Criteria for Classification of | |
|---|---|---|
| Abnormality | Laboratory TLS | Criteria for Classification of Clinical TLS |
| Hyperuricemia | Uric acid ≥8.0 mg/dL (475.8 | |
| | μmol/L) in adults or above | |
| | ULN range for age in children | |
| Hyperphosphatemia | Phosphorous >4.5 mg/dl (1.5 | |
| | mmol/L) in adults or >6.5 | |
| | mg/dL (2.1 mmol/L) in children | |
| Hyperkalemia | Potassium >6.0 mmol/L | Cardiac dysrhythmia or sudden death probably |
| | | or definitely caused by hyperkalemia |
| Hypocalcemia | Corrected calcium <7.0 mg/dL | Cardiac dysrhythmia, sudden death, seizure, |
| | (1.75 mmol/L) or ionized | neuromuscular irritability (tetany, paresthesias, |
| | calcium <4.5 mg/dl (<1.12 | carpopedal spasm, Trousseau's sign, |
| | mmol/L) | Chvostek's sign, laryngospasm, or |
| | | bronchospasm), hypotension, or heart failure |
| | | probably or definitely caused by hypocalcemia |
| Acute kidney injury | Not applicable | Increase of 0.3 mg/dL (26.5 μmol/L) in serum |
| | | creatinine level (or a single value >1.5 × age- |
| | | appropriate ULN range if no baseline creatinine |
| | | measurement is available) or the presence of |
| | | oliguria, defined as an average urine output of |
| | | <0.5 mL/kg/hour for 6 hours |

TLS = tumor lysis syndrome;
ULN = upper limit of normal.
Note:
TLS should be graded according to the National Cancer Institute Common Terminology Criteria for Adverse Events, Version 5.0.

In laboratory TLS, two or more metabolic abnormalities must be present during the same 24-hour period within 3 days before the start of therapy or up to 7 days afterward. Clinical TLS requires the presence of laboratory TLS plus an increased creatinine level, seizures, cardiac dysrhythmia, or death. Acute kidney injury is defined as an increase of 0.3 mg/dL (26.5 μmol/L) in creatinine level or a period of oliguria lasting 6 or more hours. By definition, if acute kidney injury is present, the patient has clinical TLS (Levin et al. Am. J. Kidney Dis. 50(1):1-4, 2007).

As mosunetuzumab and lenalidomide have the potential for B-cell killing, the potential risk of TLS in all patients must be considered, along with the need for prophylaxis for TLS prior to the initiation of mosunetuzumab.

All patients receive prophylaxis for TLS prior to study drug administration during dosing cycles 1 and 2. Prophylaxis guidelines include the following:
Hydration, consisting of a fluid intake of approximately 2-3 L/day starting 24-48 hours prior to the first dose of mosunetuzumab
   If a patient is hospitalized for the administration of study treatment, IV hydration at a rate of 150-200 mL/hour should begin at the conclusion of mosunetuzumab administration and continue for at least 24 hours thereafter.
   If a patient receives study treatment in the outpatient setting, fluid intake should be maintained at approximately 2-3 L/day for at least 24 hours after mosunetuzumab administration.
   Modification of fluid rate should be considered for individuals with specific medical needs.
Administration of an agent to reduce uric acid:
   Allopurinol (e.g., 300 mg/day orally beginning 72 hours prior to dose and continuing for 3-7 days afterward) should be administered at the discretion of the investigator.
   For patients with elevated uric acid levels prior to study treatment or considered to be at high risk for TLS: rasburicase (e.g., 0.2 mg/kg IV over 30 minutes prior to first dose mosunetuzumab and daily for up to 5 days thereafter) should be administered, unless contraindicated (Elitek® [rasburicase] U.S. Package Insert).
   Treatment with allopurinol/rasburicase should continue as specified above, or if laboratory evidence of TLS is observed until normalization of serum uric acid or other laboratory parameters.

Patients at high risk for TLS should continue to receive prophylaxis with allopurinol or rasburicase and adequate hydration with each subsequent dose of mosunetuzumab and lenalidomide until the patient is no longer considered to be at risk for TLS. Patients who develop either clinical or laboratory TLS during Cycle 1 should be considered for hospitalization during subsequent cycles for optimum hydration and monitoring.

If the Howard criteria for TLS (see Table 3 above) are fulfilled at any time during the study (two or more electrolyte laboratory abnormalities present simultaneously) or if there is a medically relevant laboratory abnormality in TLS-related parameters or a sign of clinical TLS (e.g., increased serum creatinine or cardiac dysrhythmia), study treatment should be withheld and patients should be hospitalized and adequately treated until normalization of laboratory abnormalities before study treatment is restarted.

3. Dosing Regimens with Acceptable Safety Profiles

In one aspect, the invention features a method of treating a subject having a previously untreated follicular lymphoma (FL), the method comprising administering to the subject mosunetuzumab and lenalidomide according to a dosing regimen comprising a first 21-day (±1 day) dosing cycle and a second 28-day (±1 day) dosing cycle, wherein: (a) the first dosing cycle comprises a first dose (C1D1), a second dose (C1D2), and a third dose (C1D3) of mosunetuzumab subcutaneously administered on Days 1, 8 (±1 day), and 15 (±1 day), respectively, of the first dosing cycle, wherein the C1D1 is about 5 mg (e.g., 5 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, or ±1 mg; e.g., 5 mg), the C1D2 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and the C1D3 is about mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), (b) the second dosing cycle comprises a single dose (C2D1) of mosunetuzumab subcutaneously administered on Day 1 of the second dosing cycle, wherein the C2D1 is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg), and (c) the second dosing cycle further comprises orally administering between about 5 mg and about 20 mg (e.g., between about 5 mg and about 10 mg, between about 10 mg and about 15 mg, between about 15 mg and about 20 mg, or between about 5 mg and about mg; e.g., about 5, 6, 7, 8, 9, 10, 11, 12, 13, 14, 15, 16, 17, 18, 19, or 20 mg) lenalidomide daily on Days 1-21 of the second dosing cycle. In some embodiments, the first dosing cycle is a three-dose dosing cycle (i.e., comprising three doses of mosunetuzumab).

In some embodiments, the dosing regimen comprises one or more (e.g., 1, 2, 3, 4, 5, 6, 7, 8, 9, 10, or more) additional dosing cycles. In some embodiments, the dosing regimen comprises one to ten (e.g., 1, 2, 3, 4, 5, 6, 7, 8, 9, or 10) additional dosing cycles. In some embodiments, the dosing regimen comprises ten additional dosing cycles. In some embodiments, the length of any of the one or more additional dosing cycles is about 28 days (±1 day). In some embodiments, the length of each of the one or more additional dosing cycles is about 28 days (±1 day).

In some embodiments, any of the one or more additional dosing cycles comprises an additional single dose of mosunetuzumab. In some embodiments, each of the one or more additional dosing cycles comprises an additional single dose of mosunetuzumab. In some embodiments, the method comprises subcutaneously administering to the subject each additional single dose of mosunetuzumab on Day 1 of each of the one or more additional dosing cycles. In some embodiments, the additional single dose of mosunetuzumab is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg). In some embodiments, each additional single dose of mosunetuzumab is about 45 mg (e.g., 45 mg±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, or ±9 mg; e.g., 45 mg).

In some embodiments, lenalidomide is not administered during the first dosing cycle. In some embodiments, lenalidomide is orally administered during any of the one or more (e.g., 1, 2, 3, 4, 5, 6, 7, 8, 9, 10, or more) additional dosing cycles. In some embodiments, lenalidomide is orally administered during any of the ten additional dosing cycles. In some embodiments, lenalidomide is orally administered during each of the ten additional dosing cycles. In some embodiments, lenalidomide is orally administered on Days 1-21 of each of the additional dosing cycles comprising administration of lenalidomide. In some embodiments, lenalidomide is not administered on the last 7 days (±1 day) of any dosing cycle comprising administration of lenalidomide. In some embodiments, lenalidomide is administered at a dose of about 10 mg (e.g., 10 mg±0.025 mg, ±0.05 mg, ±0.075 mg, ±0.1 mg, ±0.2 mg, ±0.3 mg, ±0.4 mg, ±0.5 mg, ±0.75 mg, ±1 mg, or ±2 mg; e.g., 10 mg). In some embodiments, lenalidomide is administered at a dose of about 20 mg (e.g., 20 mg±0.05 mg, ±0.1 mg, ±0.2 mg, ±0.3 mg, ±0.4 mg, ±0.5 mg, ±0.75 mg, ±1 mg, ±1.5 mg, ±2 mg, ±3 mg, or ±4 mg; e.g., 20 mg).

In some embodiments, the first dosing cycle further comprises administration of a corticosteroid. In some embodiments, the second dosing cycle further comprises administration of a corticosteroid. In some embodiments, any of the one or more additional dosing cycles comprises administration of a corticosteroid. In some embodiments, a single dose of the corticosteroid is administered to the subject prior to the administration of any dose of mosunetuzumab. In some embodiments, the corticosteroid comprises dexamethasone or methylprednisolone. In some embodiments, the corticosteroid is administered intravenously or orally. In some embodiments, the corticosteroid comprises dexamethasone and is administered at a dose of about 20 mg (e.g., 20 mg±0.05 mg, ±0.1 mg, ±0.2 mg, ±0.3 mg, ±0.4 mg, ±0.5 mg, ±0.75 mg, ±1 mg, ±1.5 mg, ±2 mg, ±3 mg, or ±4 mg; e.g., 20 mg). In some embodiments, the corticosteroid comprises methylprednisolone and is administered at a dose of about 80 mg (e.g., 80 mg±0.01 mg, ±0.025 mg, ±0.05 mg, ±0.075 mg, ±0.1 mg, ±0.2 mg, ±0.3 mg, ±0.4 mg, ±0.5 mg, ±0.75 mg, ±1 mg, ±1.5 mg, ±2 mg, ±3 mg, ±4 mg, ±5 mg, ±6 mg, ±7 mg, ±8 mg, ±10 mg, ±12 mg, ±14 mg, or ±16 mg; e.g., 80 mg).

In some embodiments, the first dosing cycle further comprises administration of an antihistamine. In some embodiments, the second dosing cycle further comprises administration of an antihistamine. In some embodiments, any of the one or more additional dosing cycles comprises administration of an antihistamine. In some embodiments, a single dose of the antihistamine is administered to the subject prior to (e.g., 30, 35, 40, 45, 50, 60 70, 80, 90, 120, 150, 180 minutes, or more prior to) the administration of any dose of mosunetuzumab. In some embodiments, the antihistamine is administered to the subject at least 30 minutes (e.g., 30, 35, 40, 45, 50, 60 70, 80, 90, 120, 150, 180 minutes, or more) prior to the administration of any dose of mosunetuzumab. In some embodiments, the antihistamine is administered orally or intravenously. In some embodiments, the antihistamine comprises diphenhydramine hydrochloride and is administered at a dose of about 50-100 mg (e.g., 50-90 mg, 50-80 mg, 50-70 mg, 50-60 mg, 60-100 mg, 70-100 mg, 80-100 mg, 90-100 mg, 70-mg, 60-90 mg, 60-80 mg, 70-90 mg, or 65-85 mg; e.g., about 50 mg, about 55 mg, about 60 mg, about mg, about 70 mg, about 75 mg, about 80 mg, about 85 mg, about 90 mg, about 95 mg, or about 100 mg).

In some embodiments, the first dosing cycle further comprises administration of an anti-pyretic. In some embodiments, the second dosing cycle further comprises administration of an anti-pyretic. In some embodiments, any of the one or more additional dosing cycles comprises administration of an anti-pyretic. In some embodiments, a single dose of the anti-pyretic is administered to the subject prior to the administration of any dose of mosunetuzumab. In some embodiments, the anti-pyretic is administered to the subject at least 30 minutes (e.g., 30, 35, 40, 45, 50, 70, 80, 90, 120, 150, 180 minutes, or more) prior to the administration of any dose of mosunetuzumab. In some embodiments, the anti-pyretic is administered orally. In some embodiments, the anti-pyretic comprises acetaminophen and is administered at a dose of about 500-1000 mg (e.g., 500-900 mg, 500-800 mg, 500-700 mg, 500-600 mg, 600-1000 mg, 700-1000 mg, 800-1000 mg, 900-1000 mg, 700-800 mg, 600-900 mg, 600-800 mg, 700-900 mg, or 650-850 mg; e.g., about 500 mg, about 550 mg, about 600 mg, about 650 mg, about 700 mg, about 750 mg, about 800 mg, about 850 mg, about 900 mg, about 950 mg, or about 1000 mg).

In some embodiments, the first dosing cycle further comprises administration of an initial dose of a prophylactic agent against tumor lysis syndrome (TLS). In some embodiments, the second dosing cycle further comprises administration of an initial dose of a prophylactic agent against TLS. In some embodiments, any of the one or more additional dosing cycles comprises administration of an initial dose of a prophylactic agent against TLS. In some embodiments, the initial dose of the prophylactic agent against TLS is administered to the subject prior to the administration of any dose of mosunetuzumab. In some embodiments, the prophylactic agent against TLS comprises allopurinol. In some embodiments, the initial dose of allopurinol is administered about 72 hours (e.g., 72±0.5 hours, ±1 hours, ±2 hours, ±3 hours, ±4 hours, ±8 hours, ±12 hours, or ±16 hours; e.g., 72 hours) prior to the administration of any dose of mosunetuzumab. In some embodiments, additional single doses of allopurinol are administered daily for 6-10 days (±1 day) after the administration of the initial dose. In some embodiments, the initial dose of allopurinol is about 300 mg (e.g., 300 mg±5 mg, ±10 mg, ±15 mg, ±20 mg, ±25 mg, ±30 mg, ±45 mg, or ±60 mg; e.g., 300 mg). In some embodiments, each additional single dose of allopurinol is about 300 mg (e.g., 300 mg±5 mg, ±10 mg, ±15 mg, ±20 mg, ±25 mg, ±30 mg, ±45 mg, or ±60 mg; e.g., 300 mg). In some embodiments, allopurinol is administered orally. In some embodiments, the prophylactic agent against TLS comprises rasburicase. In some embodiments, the initial dose of rasburicase is administered about 30 minutes (e.g., 30±0.5 minutes, 1 minutes, ±2 minutes, ±3 minutes, ±4 minutes, ±5 minutes, or ±6 minutes; e.g., 30 minutes) prior to the administration of any dose of mosunetuzumab. In some embodiments, additional single doses of rasburicase are administered daily for 1-5 days (±1 day) after the administration of the initial dose. In some embodiments, the initial dose of rasburicase is about 0.2 mg/kg (e.g., 0.2±0.005 mg/kg, ±0.01 mg/kg, ±0.02 mg/kg, ±0.03 mg/kg, or ±0.04 mg/kg; e.g., 0.2 mg/kg). In some embodiments, each additional single dose of rasburicase is about 0.2 mg/kg (e.g., 0.2±0.005 mg/kg, ±0.01 mg/kg, ±0.02 mg/kg, ±0.03 mg/kg, or ±0.04 mg/kg; e.g., 0.2 mg/kg). In some embodiments, rasburicase is administered intravenously.

In some embodiments, an IL-6R antagonist may be administered to the subject treated with the methods of the invention, e.g., when the subject exhibits CRS, e.g., after being administered any dose of mosunetuzumab. In some embodiments, the IL-6R antagonist is tocilizumab. In some embodiments, tocilizumab is administered to the subject as a single dose of about 8 mg/kg (e.g., 8 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, ±1 mg/kg, ±1.5 mg/kg, or ±2 mg/kg; e.g., 8 mg/kg), and wherein the single dose does not exceed 800 mg. In some embodiments, tocilizumab is administered to the subject as a single dose of about 12 mg/kg (e.g., 12 mg/kg±0.01 mg/kg, ±0.025 mg/kg, ±0.05 mg/kg, ±0.075 mg/kg, ±0.1 mg/kg, ±0.2 mg/kg, ±0.3 mg/kg, ±0.4 mg/kg, ±0.5 mg/kg, ±0.75 mg/kg, ±1 mg/kg, ±1.5 mg/kg, or ±2 mg/kg; e.g., 12 mg/kg), and wherein the single dose does not exceed 800 mg. In some embodiments, tocilizumab is administered intravenously.

The methods described herein may result in an acceptable safety profile for subjects having previously untreated follicular lymphoma (FL) being treated with combination therapy of mosunetuzumab and lenalidomide. In some instances, treatment using the methods described herein that include subcutaneously administering mosunetuzumab in the context of dose-escalation dosing regimen and oral administration of lenalidomide results in a reduction (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater; e.g., between 20% and 100%, between 20% and 90%, between 20% and 80%, between 20% and 70%, between 20% and 60%, between 20% and 50%, between 20% and 40%, between 20% and 30%, between 40% and 100%, between 60% and 100%, between 80% and 100%, between 30% and 70%, between 40% and 60%, between 30% and 50%, between 50% and 80%, or between 90% and 100%; e.g., about 20%, about 25%, about 30%, about 35%, about 40%, about 45%, about 50%, about 55%, about 60%, about 65%, about 70%, about 75%, about 80%, about 85%, about 90%, about 95%, about 97%, about 99%, or about 100%) or complete inhibition (100% reduction) of undesirable adverse events, such as cytokine-driven toxicities (e.g., cytokine release syndrome (CRS)), infusion-related reactions (IRRs), macrophage activation syndrome (MAS), neurologic toxicities, tumor lysis syndrome (TLS), neutropenia, thrombocytopenia, elevated liver enzymes, and/or hepatotoxicities, following treatment with mosunetuzumab using the fractionated, dose-escalation dosing regimen of the invention relative to treatment with mosunetuzumab using a non-fractioned dosing regimen.

### IV. Therapeutic Agents

### A. Mosunetuzumab

The invention provides mosunetuzumab, a bispecific antibody that binds to CD20 and CD3, useful for treating a previously untreated follicular lymphoma (FL).

Mosunetuzumab has an anti-CD20 arm having a first binding domain comprising the following six hypervariable regions (HVRs): (a) an HVR-H1 comprising the amino acid sequence of GYTFTSYNMH (SEQ ID NO: 1); (b) an HVR-H2 comprising the amino acid sequence of AIYPGNGDTSYNQKFKG (SEQ ID NO: 2); (c) an HVR-H3 comprising the amino acid sequence of VVYYSNSYWYFDV (SEQ ID NO: 3); (d) an HVR-L1 comprising the amino acid sequence of RASSSVSYMH (SEQ ID NO: 4); (e) an HVR-L2 comprising the amino acid sequence of APSNLAS (SEQ ID NO: 5); and (f) an HVR-L3 comprising the amino acid sequence of QQWSFNPPT (SEQ ID NO: 6). Mosunetuzumab comprises an anti-CD20 arm comprising a first binding domain comprising the heavy chain framework regions FR-H1, FR-H2, FR-H3, and FR-H4 comprising the sequences of SEQ ID NOs: 17-20, respectively, and the light chain framework regions FR-L1, FR-L2, FR-L3, and FR-L4 comprising the sequences of SEQ ID NOs: 21-24, respectively. Mosunetuzumab comprises an anti-CD20 arm comprising a first binding domain comprising (a) a heavy chain variable (VH) domain comprising the amino acid sequence of SEQ ID NO: 7; and (b) a light chain variable (VL) domain comprising the amino acid sequence o, SEQ ID NO: 8.

Mosunetuzumab has an anti-CD3 arm having a second binding domain comprising the following six HVRs: (a) an HVR-H1 comprising the amino acid sequence of NYYIH (SEQ ID NO: 9); (b) an HVR-H2 comprising the amino acid sequence of WIYPGDGNTKYNEKFKG (SEQ ID NO: 10); (c) an HVR-H3 comprising the amino acid sequence of DSYSNYYFDY (SEQ ID NO: 11); (d) an HVR-L1 comprising the amino acid sequence of KSSQSLLNSRTRKNYLA (SEQ ID NO: 12); (e) an HVR-L2 comprising the amino acid sequence of WASTRES (SEQ ID NO: 13); and (f) an HVR-L3 comprising the amino acid sequence of TQSFILRT (SEQ ID NO: 14). Mosunetuzumab comprises an anti-CD3 arm comprising a second binding domain comprising the heavy chain framework regions FR-H1, FR-H2, FR-H3, and FR-H4 comprising the sequences of SEQ ID NOs: 25-28, respectively, and the light chain framework regions FR-L1, FR-L2, FR-L3, and FR-L4 comprising the sequences of SEQ ID NOs: 29-32, respectively. Mosunetuzumab comprises an anti-CD3 arm comprising a second binding domain comprising (a) a VH domain comprising an amino acid sequence having the amino acid sequence of SEQ ID NO: 15; and (b) a VL domain comprising the amino acid sequence of SEQ ID NO: 16.

Mosunetuzumab has the International Nonproprietary Names for Pharmaceutical Substances (INN) List 117 (WHO Drug Information, Vol. 31, No. 2, 2017, p. 304-305), or CAS Registry No. 1905409-39-3, and having (1) an anti-CD20 arm comprising the heavy chain and light chain sequences of SEQ ID NOs: 33 and 34, respectively; and (2) an anti-CD3 arm comprising the heavy chain and light chain sequences of SEQ ID NOs: 35 and 36, respectively. Mosunetuzumab comprises (1) an anti-CD20 arm comprising a first binding domain comprising a heavy chain comprising the amino acid sequence of SEQ ID NO: 33 and a light chain comprising the amino acid sequence of SEQ ID NO: 34 and (2) an anti-CD3 arm comprising a second binding domain comprising a heavy chain comprising the amino acid sequence of SEQ ID NO: 35 and a light chain comprising the amino acid sequence of SEQ ID NO: 36.

Amino acid sequences of mosunetuzumab are summarized in Table 4 below.

TABLE 4
Sequence IDs for Mosunetuzumab

| CD3 Arm | | CD20 Arm | |
|---|---|---|---|
| SEQ ID NO: | Description | SEQ ID NO: | Description |
| 9 | CD3 HVR-H1 | 1 | CD20 HVR-H1 |
| 10 | CD3 HVR-H2 | 2 | CD20 HVR-H2 |
| 11 | CD3 HVR-H3 | 3 | CD20 HVR-H3 |
| 12 | CD3 HVR-L1 | 4 | CD20 HVR-L1 |
| 13 | CD3 HVR-L2 | 5 | CD20 HVR-L2 |
| 14 | CD3 HVR-L3 | 6 | CD20 HVR-L3 |
| 15 | CD3 VH | 7 | CD20 VH |
| 16 | CD3 VL | 8 | CD20 VL |
| 35 | CD3 heavy chain | 33 | CD20 heavy chain |
| 36 | CD3 light chain | 34 | CD20 light chain |

Mosunetuzumab may be produced using recombinant methods and compositions, for example, as described in U.S. Pat. No. 4,816,567.

### B. Lenalidomide

Lenalidomide is an immunomodulatory (IMiD) imide drug that binds to cereblon, an E3 ubiquitin ligase protein (Gribben et al. J. Clin. Oncol. 33(25):2803-2811, 2015). The immunomodulatory activity of lenalidomide is not completely understood; however, lenalidomide has been shown to enhance CD4+ and CD8+ T-cell co-stimulation, induce T-cell proliferation, and enhance IL-2 and IFN-γ (Haslett et al. J. Exp. Med. 187(11):1885-1892, 1998; Davies et al. Blood 98(1):210-216, 2001).

Lenalidomide has the CAS Registry Number 191732-72-6 and IUPAC name (3RS)-3-(4-Amino-1-oxo-1,3-dihydro-2H-isoindol-2-yl)piperidine-2,6-dione. Lenalidomide is also known by tradenames including REVLIMID®, linamid, and lenalid. Lenalidomide has the DrugBank Accession Number DB00480, PubChem CID 216326, and chemical formula C₁₃H₁₃N₃O₃.

### C. Additional Therapeutic Agents

In some instances, the methods described herein include administering mosunetuzumab and lenalidomide in combination with one or more additional therapeutic agents.

In some instances, the one or more additional therapeutic agents may prevent, reduce the rate of, or reduce the severity of cytokine release syndrome (CRS) and/or symptoms associated with CRS. In particular instances, the additional therapeutic agent used to reduce the rate or severity of CRS or prevent symptoms associated with CRS is a corticosteroid (e.g., dexamethasone (CAS #: 50-02-2), prednisone (CAS #: 53-03-2), prednisolone (CAS #: 50-42-8), or methylprednisolone (CAS #: 83-43-2)), antihistamine (e.g., diphenhydramine (CAS #: 58-73-1) and pharmaceutically acceptable salts thereof, e.g., diphenhydramine hydrochloride (CAS #: 147-24-0)) or an interleukin-6 receptor (IL-6R) antagonist (e.g., tocilizumab (CAS #: 375823-41-9), sarilumab (CAS #: 1189541-98-7), vobarilizumab (ALX-0061; CAS #: 1628814-88-9), satralizumab (SA-237; CAS #: 1535963-91-7), and alternatives thereof). In some instances, the additional therapeutic agent is a corticosteroid. In particular instances, the corticosteroid is dexamethasone or methylprednisolone. In a particular instance, the antihistamine is diphenhydramine hydrochloride. In a particular instance, the anti-pyretic is acetaminophen (i.e., paracetamol) or pharmaceutically acceptable salts thereof. In a particular instance, the IL-6R antagonist is tocilizumab.

In some instances, the one or more additional therapeutic agents may be used in the treatment of neutropenia. In some instances, the additional therapeutic agents may be prevent symptoms associated with neutropenia. In some instances, the additional therapeutic agents may reduce the rate or severity of neutropenia. In particular instances, the additional therapeutic agent is granulocyte colony-stimulating factor (G-CSF or GCSF) or colony-stimulating factor 3 (CSF 3). The mRNA sequence of human G-CSF/CSF 3 includes, e.g., NCBI RefSeq No. NM_000759, NM_001178147, NM_172219, and NM_172220, and the protein amino acid sequence of human G-CSF/CSF 3 includes, e.g., NCBI RefSeq No. NP_000750, NP_001171618, NP_757373, and NP_757374.

In some instances, the one or more additional therapeutic agents is a prophylactic agent against, e.g., to prevent, reduce the rate of, or reduce the severity of tumor lysis syndrome (TLS). In some instances, the prophylactic agent against TLS is allopurinol (CAS #: 315-30-0), rasburicase (Elitek™; CAS #: 134774-45-1), or suitable alternatives and/or pharmaceutically acceptable salts thereof. In some instances, the prophylactic agent against TLS reduces serum uric acid (e.g., reduces elevated uric acid levels, e.g., relative to a baseline or reference value).

In some instances, additional therapeutic agents useful in the present invention include therapeutic antibodies, such as alemtuzumab (CAMPATHCI), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (BEXXAR®, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, tafasitamab, talizumab, tefibazumab, tocilizumab, toralizumab, tucotuzumab celmoleukin, tucusituzumab, umavizumab, urtoxazumab, ustekinumab, visilizumab, and briakinumab.

### IV. Pharmaceutical Compositions and Formulations

Mosunetuzumab, lenalidomide, and other therapeutic agents described herein can be used in pharmaceutical compositions and formulations. Pharmaceutical compositions and formulations of mosunetuzumab, lenalidomide, and/or other agents describe herein (e.g., corticosteroids, antihistamines, anti-pyretics, prophylactic agents against TLS, and/or IL-6R antagonists) can be prepared by mixing one, two, three, or more agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. Corticosteroids, antihistamines, anti-pyretics, prophylactic agents against TLS, and/or IL-6R antagonists may also be formulated according to standard formulation and/or manufacturing practices. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methyl methacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules.

The formulations to be used for in vivo administration are generally sterile. Sterility may be readily accomplished, e.g., by filtration through sterile filtration membranes.

In some embodiments, mosunetuzumab is formulated for administration subcutaneously. In some embodiments, lenalidomide is formulated for administration orally. In some embodiments, dexamethasone is formulated for administration orally or intravenously. In some embodiments, methylprednisolone is formulated for administration orally or intravenously. In some embodiments, allopurinol is formulated for administration orally. In some embodiments, rasburicase is formulated for administration intravenously. In some embodiments, tocilizumab is formulated for administration intravenously.

### V. Kits and Articles of Manufacture

In another aspect of the invention, a kit or an article of manufacture containing materials useful for the treatment, prevention, and/or diagnosis of the disorders described above is provided. The kit or article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be a vial having a stopper pierceable by a hypodermic injection needle). At least one active agent in the composition is mosunetuzumab or lenalidomide described herein. The label or package insert indicates that the composition is used for treating previously untreated follicular lymphoma (FL) and further includes information related to at least one of the dosing regimens described herein. In some embodiments, the label or package insert indicates that the composition is used for treating previously untreated FL in a patient. Moreover, the kit or article of manufacture may comprise (a) a first container with a composition contained therein, wherein the composition comprises mosunetuzumab, lenalidomide, or both mosunetuzumab and lenalidomide; and (b) a second container with a composition contained therein, wherein the composition comprises a further cytotoxic or otherwise therapeutic agent. Alternatively, or additionally, the kit or article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLE

The following is an example of methods and compositions of the invention. It is understood that various other embodiments may be practiced, given the general description provided above.

### Example 1. A Phase Ib/II, Open-Label, Non-Randomized, Multicenter Study Evaluating the Safety, Tolerability, and Pharmacokinetics of Subcutaneous Mosunetuzumab in Combination with Lenalidomide in Patients with Previously Untreated Follicular Lymphoma

### Study Design

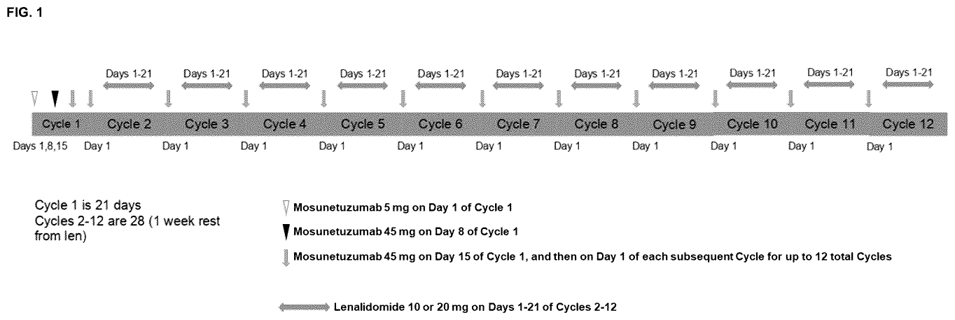

Study CO41942 is an ongoing Phase Ib/II, open-label, multicenter study, and includes evaluation of the safety, tolerability, and pharmacokinetics of subcutaneous mosunetuzumab administration in combination with lenalidomide in patients with previously untreated follicular lymphoma (FL). The study design of the present study is summarized in FIG. 1.

The study includes an expansion cohort of about 20-40 patients who are dosed with the recommended Phase 2 dosing (RP2D) of mosunetuzumab.

Study treatment is administered for 12 cycles; the duration of dosing cycle (Cycle) 1 is 21 days, and the duration of Cycle 2-12 is 28 days. Mosunetuzumab is administered together with lenalidomide in Cycle 2 after patients complete Cycle 1 step-up dosing.

### Inclusion Criteria

Age 18 years
Eastern Cooperative Oncology Group (ECOG) Performance Status of 0, 1, or 2 (see Table below)
Previously untreated patients with follicular lymphoma (FL) must require systemic therapy assessed by investigator based on Groupe d'Etude des Lymphomes Folliculaires (GELF) criteria (Brice et al. J Clin Oncol. 15(3):1110-1117, 1997)
Histologically documented FL of Grade 1, 2, or 3a (Swerdlow S H, et al. Blood 2016; 127:2375-90), and that expresses CD20
Fluorodeoxyglucose-avid lymphoma (i.e., positron emission tomography (PET)-positive lymphoma)
At least one bi-dimensionally measurable nodal lesion (>1.5 cm in its largest dimension by PET-computed tomography (CT) scan), or at least one bi-dimensionally measurable extranodal lesion (>1.0 cm in its largest dimension by PET-CT scan)
Adequate hematologic function (without growth factors or blood product transfusion within 14 days of first dose of study drug administration) is defined as follows:
   Hemoglobin ≥9 g/dL
   Absolute neutrophil count (ANC)≥1.0×10⁹/L
   Platelet count ≥75×10⁹/L
Measured or estimated creatinine clearance 50 mL/min by institutional standard method
Aspartate aminotransferase (AST) or alanine transaminase (ALT)<2.5×upper limit of normal (ULN)
Serum total bilirubin <1.5×ULN (or <3×ULN for patients with Gilbert syndrome)
For women of childbearing potential: agreement to remain abstinent (refrain from heterosexual intercourse) or use two adequate methods of contraception, including at least one method with a failure rate of <1% per year, for at least 28 days prior to Day 1 of Cycle 1, during the treatment period (including periods of treatment interruption), and for at least 28 days after the last dose of lenalidomide, 3 months after the final dose of tocilizumab, and 3 months after the final dose of mosunetuzumab. Women must refrain from donating eggs during this same period.
For men: agreement to remain abstinent (refrain from heterosexual intercourse) or use contraceptive measures and agreement to refrain from donating sperm.

TABLE 5
ECOG Performance Status Scale

| Grade | Description |
|---|---|
| 0 | Fully active; able to carry on all pre-disease performance |
| | without restriction. |
| 1 | Restricted in physically strenuous activity but ambulatory and |
| | able to carry out work of a light or sedentary nature |
| | (e.g., light housework or office work). |
| 2 | Ambulatory and capable of all self-care but unable to carry |
| | out any work activities. Up and about >50% of waking hours. |
| 3 | Capable of only limited self-care; confined to a bed or chair |
| | >50% of waking hours. |
| 4 | Completely disabled. Cannot carry on any self-care. |
| | Totally confined to bed or chair. |
| 5 | Dead. |

### Exclusion Criteria

Any history of Grade 3b FL
Any history of transformation and/or diffuse large B cell lymphoma (DLBCL)
Active or history of central nervous system (CNS) lymphoma or leptomeningeal infiltration
Documented refractoriness to lenalidomide, defined as no response (PR or CR) within 6 months of therapy
Prior standard or investigational anti-cancer therapy as specified below:
   Lenalidomide exposure within 12 months prior to Day 1 of Cycle 1
   Fludarabine or alemtuzumab within 12 months prior to Day 1 of Cycle 1
   Radioimmunoconjugate within 12 weeks prior to Day 1 of Cycle 1
   Prior anti-lymphoma treatment with monoclonal antibody or antibody-drug conjugate within 4 weeks prior to Day 1 of Cycle 1
   Treatment with any chemotherapeutic agent, or treatment with any other anti-cancer agent (investigational or otherwise) within 4 weeks or 5 half-lives of the drug, whichever is shorter, prior to first dose of study treatment
Clinically significant toxicity (other than alopecia) from prior treatment that has not resolved to Grade 2 (per NCI CTCAE v5.0) prior to Day 1 of Cycle 1
Known history of idiopathic pulmonary fibrosis, organizing pneumonia (e.g., bronchiolitis obliterans), drug-induced pneumonitis or evidence of active pneumonitis on screening chest CT scan
Treatment with systemic immunosuppressive medications, including, but not limited to, prednisone, azathioprine, methotrexate, thalidomide, and anti-tumor necrosis factor agents within 2 weeks prior to Day 1 of Cycle 1
   The use of inhaled corticosteroids is permitted.
   The use of mineralocorticoids for management of orthostatic hypotension is permitted.
   The use of physiologic doses of corticosteroids for management of adrenal insufficiency is permitted.
   If corticosteroid treatment is urgently required for lymphoma symptom control prior to the start of study treatment, 100 mg of prednisone or equivalent can be given for a maximum of 5 days.
History of solid organ transplantation
History of severe allergic or anaphylactic reaction to humanized, chimeric or murine monoclonal antibodies
Known hypersensitivity to biopharmaceuticals produced in Chinese hamster ovary cells or any component of the mosunetuzumab, lenalidomide, or thalidomide formulation, including mannitol
History of erythema multiforme, Grade 3 rash, or blistering following prior treatment with immunomodulatory derivatives
Known active bacterial, viral, fungal, or other infection, or any major episode of infection requiring treatment with IV antibiotics within 4 weeks of Day 1 of Cycle 1
Known or suspected chronic active Epstein-Barr virus (EBV) infection
Known or suspected hemophagocytic syndrome
Active hepatitis B infection: patients who are hepatitis B surface antigen (HBsAg) negative and hepatitis B core antibody (HBcAb) positive, must be negative for hepatitis B virus (HBV) polymerase chain reaction (PCR) to be eligible for study participation.
Active hepatitis C infection: patients who are positive for hepatitis C virus (HCV) antibody must be negative for HCV by PCR to be eligible for study participation.
Known history of human immunodeficiency virus (HIV) positive status: for patients with unknown HIV status, HIV testing is performed at screening if required by local regulations.
History of progressive multifocal leukoencephalopathy (PML)
Administration of a live, attenuated vaccine within 4 weeks before first dose of study treatment or anticipation that such a live attenuated vaccine is required during the study
   Patients must not receive live, attenuated vaccines (e.g., FluMist®) while receiving study treatment or after the last dose until B-cell recovery to the normal ranges. Killed vaccines or toxoids should be given at least 4 weeks prior to the first dose of study treatment to allow development of sufficient immunity.
   Approved non-live COVID-19 vaccine is permitted
   Inactivated influenza vaccination should be given during influenza season only.
Other malignancy that could affect compliance with the protocol or interpretation of results, with the exception of the following:
   Any of the following malignancies previously curatively treated: carcinoma in situ of the cervix, good-prognosis ductal carcinoma in situ of the breast, basal, or squamous cell skin cancer
   Stage I melanoma, low-grade, early-stage localized prostate cancer, or any other previously treated malignancy that has been in remission without treatment for 2 years prior to enrollment
   Active autoimmune disease requiring treatment
   Patients with a history of autoimmune-related hypothyroidism on a stable dose of thyroid-replacement hormone may be eligible.
   Patients with controlled Type 1 diabetes mellitus who are on an insulin regimen are eligible for the study.
   Patients with a history of disease-related immune thrombocytopenic purpura, or autoimmune hemolytic anemia, or other stable autoimmune diseases may be eligible.
   Patients with a history of autoimmune disease, including, but not limited to, myasthenia gravis, myositis, autoimmune hepatitis, systemic lupus erythematosus, rheumatoid arthritis, inflammatory bowel disease, vascular thrombosis associated with antiphospholipid syndrome, Wegener granulomatosis, Sjögren syndrome, Guillain-Barré syndrome, multiple sclerosis, vasculitis, or glomerulonephritis, with treatment-free interval from immunosuppressive therapy for 12 months, may be eligible.
Prior allogeneic hematopoietic stem cell transplant
Grade ≥2 neuropathy
Evidence of any significant, uncontrolled concomitant disease that could affect compliance with the protocol or interpretation of results, including, but not limited to significant cardiovascular disease (e.g., New York Heart Association Class III or IV cardiac disease, myocardial infarction within the previous 6 months, unstable arrhythmia, or unstable angina) or significant pulmonary disease (such as obstructive pulmonary disease or history of bronchospasm)
Major surgical procedure other than for diagnosis within 28 days prior to Day 1 of Cycle 1 or anticipation of a major surgical procedure during the course of the study
Clinically significant history of liver disease, including viral or other hepatitis, current alcohol abuse, or cirrhosis
Pregnant or lactating or intending to become pregnant during the study

### Study Treatments

Mosunetuzumab Administration

Mosunetuzumab is administered subcutaneously (SC) following a step-up dosing regimen: in Cycle 1 (21-day dosing cycle), patients receive 5 mg on Day 1 (C1D1 dose); 45 mg on Day 8 (C1D2 dose); and 45 mg on Day 15 (C1D3 dose). In Cycles 2-12 (28-day dosing cycles), patients receive 45 mg on Day 1 (C2D1-C12D1 doses). Hospitalization is not required for patients who receive subcutaneous mosunetuzumab (based on prior clinical experience in Study G029781) unless clinically indicated.

In the event that a patient has a toxicity necessitating SC mosunetuzumab interruption for >7 days prior to the Cycle 1 Day 8 dose, the patient is required to repeat the 5 mg dose prior to resuming the planned treatment schedule (7 days after the administration of the 5 mg dose). If dose delay results in a treatment-free interval of 6 weeks or longer, step up dosing of SC mosunetuzumab is required on Days 1 (5 mg), and 8 (45 mg) for the first cycle after the dose delay. Corticosteroid prophylaxis should be administered on days when repeat doses are administered to mitigate cytokine release syndrome (CRS) risks.

Corticosteroid premedication consisting of dexamethasone 20 mg or methylprednisolone 80 mg is administered orally or intravenously (IV) prior to the administration of each mosunetuzumab dose. In addition, premedication with oral or IV analgesic/antipyretic (e.g., 500-1,000 mg acetaminophen or paracetamol) and/or with oral or IV antihistamine (e.g., 50-100 mg diphenhydramine) is administered per standard institutional practice prior to administration of mosunetuzumab. Premedication with analgesic/antipyretic and antihistamine occurs at least 30 minutes prior to administration of mosunetuzumab. Premedication is administered to all patients in Cycle 1, and may be optional from Cycle 2 and beyond. However, if a patient experiences CRS in earlier doses, premedication with corticosteroids is administered for subsequent doses.

Additionally, for patients at risk of tumor lysis syndrome (TLS; e.g., because of bulky disease or renal impairment (creatinine clearance <60 mL/min)), allopurinol or a suitable alternative such as rasburicase is used as premedication.

Lenalidomide Administration

Lenalidomide is administered orally (PO) 20 mg once daily on Days 1-21 of Cycles 2-12 (28-day cycles). Lenalidomide is not administered on the last 7 days of each of the 28-day Cycles 2-12. If creatinine clearance is <60 mL/min, lenalidomide is instead started at a dose of 10 mg/day.

On the days when lenalidomide is administered with mosunetuzumab, lenalidomide is administered first, followed by subcutaneous mosunetuzumab injection. Lenalidomide is administered at approximately the same time each day. If a dose of lenalidomide is missed and it has been ≤12 hours since the time of the scheduled dose, the patient is allowed to take the missed dose. If it has been >12 hours, the dose is skipped and the next dose is taken at the regularly scheduled time. Two doses are not to be taken at the same time. If a dose is vomited, the dose is not re-taken.

Granulocyte colony-stimulating factor (G-CSF) may be administered per American Society of Clinical Oncology, European Organisation [sic] for Research and Treatment of Cancer (EORTC), and European Society for Medical Oncology guidelines (Smith et al. J. Clin. Oncol. 33(28):3199-3212; 2015).

Prophylactic treatment with antibiotics should be administered as per standard practice.

Lenalidomide increases the risk of thromboembolism (TE). Anticoagulation prophylaxis may be given after a careful assessment of a patient's underlying risk factors. All patients are recommended to receive daily low-dose aspirin (81-100 mg) during lenalidomide treatment and until 28 days after the last dose of lenalidomide. Patients who are unable to tolerate aspirin, who have a history of TE, and who are at high risk of TE may receive warfarin or low-molecular-weight heparin or novel oral anticoagulant in accordance with institutional practice.

Tocilizumab Administration

Tocilizumab is administered when necessary to patients who experience a CRS event. Tocilizumab is administered at a dose of 8 mg/kg IV (for participants at or above 30-kg weight only) or 12 mg/kg (for participants less than 30 kg weight) for maximum of 4 doses; doses exceeding 800 mg per infusion are not recommended.

Dose Modification

Mosunetuzumab dosing is not modified in this study. Patients receiving mosunetuzumab who experience a Grade 4, related, non-hematologic adverse event discontinue study treatment.

Lenalidomide dosing is modified for patients individually based on toxicity rules described below and further in detail in Table 6. The lenalidomide dose may be reduced in 5-mg increments (e.g., 20 mg reduced to 15 mg; 15 mg reduced to 10 mg, 10 mg reduced to 5 mg). There is no more than one dose reduction per dosing cycle. If the lenalidomide dose is reduced, re-escalation is not permitted. If lenalidomide was started at a dose of 10 mg/day for creatinine clearance ≥50 but <60 mL/min, and creatinine clearance remains above 50 mL/min after 2 cycles, the lenalidomide dose can be increased to mg if the patient has tolerated therapy. Doses that were missed, due to toxicity or any other reasons, are not rescheduled or readministered.

TABLE 6
Toxicity-Based Dose Modification for Lenalidomide

| NCI CTCAE Toxicity | |
|---|---|
| Grade | Action Required |
| Grade 3 or 4 anemia^{a, b} | For patients on a lenalidomide dose ≥10 mg who have had one or |
| | no prior lenalidomide dose reductions |
| | Withhold lenalidomide and check full blood count at least every 7 days. |
| | Administer RBCs or platelets as required. |
| | If the event is ongoing on Day 1 of the next cycle but improves to |
| | Grade ≤ 2 or baseline ≤ 14 days after the scheduled date of the cycle, |
| | resume lenalidomide at current dose or reduced dose if the patient |
| | received <21 days of lenalidomide due to toxicity. |
| | If the event is ongoing on Day 1 of the next cycle but improves to |
| | Grade ≤ 2 or baseline 15-21 days after the scheduled date for the next |
| | cycle, resume lenalidomide at a reduced dose for current and |
| | subsequent cycles.^{a, b} |
| | For patients on a lenalidomide dose of 5 mg and patients who have |
| | had 3 prior lenalidomide dose reductions: |
| | Permanently discontinue lenalidomide. |
| Renal Toxicity | For patients with creatinine clearance at study entry of ≥50 but <60 |
| | mL/min, lenalidomide dosing starts at 10 mg. If patient tolerates the |
| | therapy, and creatinine clearance remains >50 ml/min after 2 cycles, |
| | the lenalidomide dose may be increased to 15 mg. |
| | For patients with creatinine clearance that decrease during treatment |
| | to above 30 but <60 mL/min, lenalidomide dose is reduced to 10 mg, |
| | dose is not re-escalated |
| | If creatinine clearance is <30 mL/min and dialysis is not required, |
| | lenalidomide should be given at a dose of 5 mg/day. |
| | If creatinine clearance is <30 mL/min and dialysis is required, |
| | lenalidomide should be given at a dose of 5 mg/day. On dialysis days, |
| | lenalidomide is administered after dialysis. |

Rash

| | |
|---|---|
| Grade 2 or Grade 3 | Hold treatment with lenalidomide (interrupt dose). |
| without desquamating | Possibly administer steroid prophylaxis. When resolved (Grade ≤ 1), |
| | restart lenalidomide at the same dose level |
| Desquamating (blistering) | Permanently discontinue lenalidomide. |
| Grade ≥ 3 or Non- | |
| desquamating Grade 4 | |

Allergic Reaction or Hypersensitivity

| | |
|---|---|
| Grade 2 | If allergic reaction has occurred on or after Day 15 hold (interrupt |
| | dose) for the rest of the cycle and follow at least every 7 days |
| | If allergic reaction has occurred before Day 15 and resolved to Grade |
| | 1 restart at same dose level for the rest of the cycle |
| | In both cases, restart subsequent cycle at next lower dose and please |
| | contact the Medical Monitor to discuss any further steroid prophylaxis |
| Grade 3-4 | Permanently discontinue lenalidomide. |

Constipation

| | |
|---|---|
| Grade 1-2 | Maintain lenalidomide at full dose |
| Grade ≥3 | If constipation has occurred on or after Day 15, hold lenalidomide |
| | (interrupt dose) for the rest of the cycle and follow at least every 7 days |
| | If constipation has occurred before Day 15 and resolved to Grade ≤ 2 |
| | restart lenalidomide at same dose level for the rest of the cycle |
| | In both cases, restart subsequent cycle at next lower dose |

Venous thrombosis/embolism

| | |
|---|---|
| Grade ≥ 3 | Hold lenalidomide (interrupt dose) and start anticoagulation; restart at |
| | Investigator's discretion (maintain dose level) |

Peripheral neuropathy

| | |
|---|---|
| Grade 2 or 3 | If peripheral neuropathy is considered likely related to lenalidomide |
| | and there is improvement to Grade ≤ 1 or baseline, resume lenalidomide |
| | at a reduced dose for current and subsequent cycles. |
| Grade 4 | Permanently discontinue lenalidomide |
| Other lenalidomide-related | If AE has occurred on or after Day 15, hold lenalidomide (interrupt |
| non-hematologic AEs | dose) for the rest of the cycle and follow at least every 7 days. |
| Grade ≥ 3 | If AE has occurred before Day 15 and resolved to Grade ≤ 2, resume |
| | lenalidomide at same dose level for the rest of the cycle. |
| | In both cases, restart subsequent cycle of lenalidomide at next lower |
| | dose. |

AE = adverse event;
NSAID = nonsteroidal anti-inflammatory drug;
TLS = tumor lysis syndrome;
ULN = upper limit of normal.
^{a}Dose modifications apply only to events that are considered to be related to lenalidomide.
^{b}Graded according to National Cancer Institute Common Terminology Criteria for Adverse Events version 5 (NCI CTCAE Version 5.0; cancer.gov).

### Prohibited Therapies

Use of the therapies including, but not limited to, those listed below is prohibited during the study:
Investigational or unlicensed/unapproved agents
Administration of live vaccines
Cytotoxic chemotherapy other than study treatments intended for treatment of lymphoma
Radiotherapy for treatment of lymphoma (except pre-planned radiotherapy)
Immunotherapy other than study treatments for treatment of lymphoma
Immunosuppressive therapy (except medications indicated per protocol, including corticosteroids and tocilizumab)
Hormone therapy (other than contraceptives, hormone-replacement therapy, or megestrol acetate).
Adjuvant endocrine therapy for non-metastatic, hormone receptor-positive breast cancer is permitted.
Biologic or targeted agents for treatment of lymphoma
Herbal therapies intended as treatment of lymphoma
Any therapies intended for the treatment of lymphoma, whether approved by local regulatory authorities or investigational

Patients who require the use of any of these agents are discontinued from study treatment. Patients who are discontinued from study treatment are followed for safety outcomes for 90 days following the patient's last dose of study treatment or until the patient receives another anti-cancer therapy, whichever occurs first.

### Safety

Safety is assessed through summaries of adverse events and summaries of changes in laboratory test results. All adverse events, serious adverse events, adverse events leading to death, adverse events of special interest, and adverse events leading to study treatment discontinuation, occurring on or after first study treatment are summarized by mapped term, appropriate thesaurus levels, and NCI CTCAE v5.0 toxicity grade (cancer.gov). All serious adverse events are listed separately and summarized.

Adverse Events (AEs)

According to the International Conference on Harmonization (ICH) guideline for Good Clinical Practice (ich.org), an adverse event is any untoward medical occurrence in a clinical investigation subject administered a pharmaceutical product, regardless of causal attribution. An adverse event can therefore be any of the following:
Any unfavorable and unintended sign (including an abnormal laboratory finding), symptom, or disease temporally associated with the use of a medicinal product, whether or not considered related to the medicinal product
Any new disease or exacerbation of an existing disease (a worsening in the character, frequency, or severity of a known condition)
Recurrence of an intermittent medical condition (e.g., headache) not present at baseline
Any deterioration in a laboratory value or other clinical test (e.g., electrocardiogram (ECG), X-ray) that is associated with symptoms or leads to a change in study treatment or concomitant treatment or discontinuation from study drug
Adverse events that are related to a protocol-mandated intervention, including those that occur prior to assignment of study treatment (e.g., screening invasive procedures such as biopsies)

Serious Adverse Events (SAEs)

A serious adverse event is any adverse event that meets any of the following criteria:
Is fatal (i.e., the adverse event actually causes or leads to death)
Is life-threatening (i.e., the adverse event places the patient at immediate risk of death)

This does not include any adverse event that, had it occurred in a more severe form or was allowed to continue, might have caused death.
Requires or prolongs inpatient hospitalization
Results in persistent or significant disability/incapacity (i.e., the adverse event results in substantial disruption of the patient's ability to conduct normal life functions)
Is a congenital anomaly/birth defect in a neonate/infant born to a mother exposed to study drug
May jeopardize the patient or may require medical/surgical intervention to prevent one of the outcomes listed above

The terms “severe” and “serious” are not synonymous. Severity refers to the intensity of an adverse event (e.g., rated as mild, moderate, or severe, or according to NCI CTCAE; cancer.gov); the event itself may be of relatively minor medical significance (e.g., severe headache without any further findings).

Severity and seriousness need to be independently assessed for each adverse event.

Adverse Events of Special Interest (AESIs)

Adverse events of special interest for this study are as follows:
Cases of potential drug-induced liver injury that include an elevated alanine transaminase (ALT) or aspartate aminotransferase (AST) in combination with either an elevated bilirubin or clinical jaundice, as defined by Hy's Law (an elevated ALT or AST (>3×upper limit of normal (ULN)) in combination with either an elevated total bilirubin (>2×ULN) or clinical jaundice in the absence of cholestasis or other causes of hyperbilirubinemia is considered to be an indicator of severe liver injury).
Suspected transmission of an infectious agent by the study drug, as defined below:

Any organism, virus, or infectious particle (e.g., prion protein transmitting transmissible spongiform encephalopathy), pathogenic or non-pathogenic, is considered an infectious agent. A transmission of an infectious agent may be suspected from clinical symptoms or laboratory findings that indicate an infection in a patient exposed to a medicinal product. This term applies only when a contamination of the study drug is suspected.

Adverse events of special interest for mosunetuzumab include the following:
Grade ≥2 cytokine release syndrome (CRS)
Grade ≥2 neurologic adverse event
Grade ≥2 injection site reaction (for subcutaneous (SC) mosunetuzumab)
Any suspected Hemophagocytic lymphohistiocytosis (HLH)
Tumor lysis syndrome (TLS; Grade ≥3 by definition)
Febrile neutropenia (minimum Grade 3 by definition)
Grade ≥2 AST, ALT, or total bilirubin elevation
Any Grade disseminated intravascular coagulation (minimum Grade 2 by definition)
Grade ≥2 tumor inflammation or flare (e.g., manifestation of signs/symptoms associated with an increase in size of known nodal or extranodal lesions by clinical or radiographic assessment, new onset, or worsening of preexisting pleural effusions)
Any Grade pneumonitis/interstitial lung disease (excluding pneumonia of infectious etiology)

Adverse events of special interest for lenalidomide include the following:
Embryo-fetal toxicity
Febrile neutropenia (minimum Grade 3 by definition)
Venous and arterial thromboembolic events
Grade ≥3 severe skin reactions
Grade ≥3 renal impairment
Grade ≥3 thyroid disorder
Grade ≥3 peripheral neuropathy
Second primary malignancies

### Efficacy Analyses

The efficacy of treatment of previously untreated FL with mosunetuzumab and lenalidomide is assessed on the basis of the following endpoints using standard criteria for NHL, assessed by the investigator using the Lugano classification (Cheson B D, et al. J Clin Oncol 2014; 32:1-9). Responses are assessed based on positron emission tomography/computer tomography (PET-CT) scans.

CRR, defined as the proportion of patients whose best overall response is a CR during the study. The exact 95% confidence intervals using the Clopper-Pearson method is provided.

ORR, defined as the proportion of patients whose best overall response is a CR or PR during the study. The exact 95% CIs using the Clopper-Pearson method is provided.

DOR, defined as the time from the first occurrence of a documented objective response to disease progression or relapse or death from any cause, whichever occurs first. The Kaplan-Meier estimate is provided. The Brookmeyer-Crowley method is used to construct the 95% CI for the median DOR. Duration of response includes all patients with a CR or PR according to the investigator.

DOCR, defined as the time from the first occurrence of a documented complete response to disease progression or relapse or death from any cause, whichever occurs first. The Kaplan-Meier estimate is provided. The Brookmeyer-Crowley method is used to construct the 95% CI for the median DOR. DOCR includes all patients with a CR according to the investigator.

### Pharmacokinetics (PK) Analyses

Individual and mean serum mosunetuzumab concentration versus time data is tabulated and plotted. The plasma PK of mosunetuzumab is summarized by estimating the Cₘₐₓ, Cₘᵢₙ, and AUC, if appropriate. These parameters are tabulated and summarized (mean, standard deviation, coefficient of variation, median, minimum, and maximum).

Additional PK analyses are conducted as appropriate. In addition, these data are analyzed using population PK modeling.

Serum concentrations of lenalidomide is measured. The concentration of lenalidomide is summarized using descriptive statistics as described above.

### Embodiments

Some embodiments of the technology described herein can be defined according to any of the following numbered embodiments:

1. A method of treating a subject having a previously untreated follicular lymphoma (FL), the method comprising administering to the subject mosunetuzumab and lenalidomide according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab subcutaneously administered on Day 1 of the second dosing cycle, wherein the C2D1 is about 45 mg, and
(c) the second dosing cycle further comprises orally administering between about 5 mg and about 20 mg lenalidomide daily on Days 1-21 of the second dosing cycle.

2. Mosunetuzumab for use in combination with lenalidomide for treating a subject having a previously untreated F), wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about 45 mg, and
(c) the second dosing cycle further comprises between about 5 mg and about 20 mg lenalidomide to be orally administered daily on Days 1-21 of the second dosing cycle.

3. Lenalidomide for use in combination with mosunetuzumab for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about mg, and
(c) the second dosing cycle further comprises between about 5 mg and about 20 mg lenalidomide to be orally administered daily on Days 1-21 of the second dosing cycle.

4. Use of mosunetuzumab in combination with lenalidomide for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about mg, and
(c) the second dosing cycle further comprises between about 5 mg and about 20 mg lenalidomide to be orally administered daily on Days 1-21 of the second dosing cycle.

5. Use of lenalidomide in combination with mosunetuzumab for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about 45 mg, and
(c) the second dosing cycle further comprises between about 5 mg and about 20 mg lenalidomide to be orally administered daily on Days 1-21 of the second dosing cycle.

6. Use of mosunetuzumab in the manufacture of a medicament for use in combination with lenalidomide for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about mg, and
(c) the second dosing cycle further comprises between about 5 mg and about 20 mg lenalidomide to be orally administered daily on Days 1-21 of the second dosing cycle.

7. Use of lenalidomide in the manufacture of a medicament for use in combination with mosunetuzumab for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about mg, and
(c) the second dosing cycle further comprises between about 5 mg and about 20 mg lenalidomide to be orally administered daily on Days 1-21 of the second dosing cycle.

8. The method, mosunetuzumab for use, lenalidomide for use, or use of any one of embodiments 1-7, wherein the dosing regimen comprises one or more additional dosing cycles.

9. The method, mosunetuzumab for use, lenalidomide for use, or use of embodiment 8, wherein the dosing regimen comprises one to ten additional dosing cycles.

10. The method, mosunetuzumab for use, lenalidomide for use, or use of embodiment 8 or 9, wherein the dosing regimen comprises ten additional dosing cycles.

11. The method, mosunetuzumab for use, lenalidomide for use, or use of any one of embodiments 8-10, wherein the length of each of the one or more additional dosing cycles is about 28 days.

12. The method, mosunetuzumab for use, lenalidomide for use, or use of any one of embodiments 8-11, wherein each of the one or more additional dosing cycles comprises an additional single dose of mosunetuzumab.

13. The method, mosunetuzumab for use, lenalidomide for use, or use of embodiment 12, wherein the additional single dose of mosunetuzumab is about 45 mg.

14. The method of embodiment 12 or 13, wherein the method comprises subcutaneously administering to the subject each additional single dose of mosunetuzumab on Day 1 of each of the one or more additional dosing cycles.

15. The mosunetuzumab for use, lenalidomide for use, or use of embodiment 12 or 13, wherein each additional single dose of mosunetuzumab is to be subcutaneously administered to the subject on Day 1 of each of the one or more additional dosing cycles.

16. The method, mosunetuzumab for use, lenalidomide for use, or use of any one of embodiments 1-15, wherein lenalidomide is not administered or is not to be administered during the first dosing cycle.

17. The method, mosunetuzumab for use, lenalidomide for use, or use of any one of embodiments 8-16, wherein lenalidomide is orally administered or is to be orally administered during any of the one or more additional dosing cycles.

18. The method, mosunetuzumab for use, lenalidomide for use, or use of any one of embodiments 10-17, wherein lenalidomide is orally administered or is to be orally administered during each of the ten additional dosing cycles.

19. The method, mosunetuzumab for use, lenalidomide for use, or use of embodiment 17 or 18, wherein lenalidomide is orally administered or is to be orally administered on Days 1-21 of each of the additional dosing cycles comprising administration of lenalidomide.

20. The method, mosunetuzumab for use, lenalidomide for use, or use of any one of embodiments 1-19, wherein lenalidomide is not administered or is not to be administered on the last 7 days of any dosing cycle comprising administration of lenalidomide.

21. The method, mosunetuzumab for use, lenalidomide for use, or use of any one of embodiments 1-20, wherein lenalidomide is administered or is to be administered at a dose of about mg.

22. The method, mosunetuzumab for use, lenalidomide for use, or use of any one of embodiments 1-21, wherein lenalidomide is administered or is to be administered at a dose of about mg.

23. A method of treating a subject having a previously untreated FL, the method comprising administering to the subject mosunetuzumab and lenalidomide according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab subcutaneously administered on Day 1 of the second dosing cycle, wherein the C2D1 is about 45 mg, and
(c) the second dosing cycle further comprises orally administering about 10 mg lenalidomide daily on Days 1-21 of the second dosing cycle.

24. Mosunetuzumab for use in combination with lenalidomide for treating a subject having a previously untreated F), wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about 45 mg, and
(c) the second dosing cycle further comprises about 10 mg lenalidomide to be orally administered daily on Days 1-21 of the second dosing cycle.

25. Lenalidomide for use in combination with mosunetuzumab for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about mg, and
(c) the second dosing cycle further comprises about 10 mg lenalidomide to be orally administered daily on Days 1-21 of the second dosing cycle.

26. Use of mosunetuzumab in combination with lenalidomide for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about mg, and
(c) the second dosing cycle further comprises about 10 mg lenalidomide to be orally administered daily on Days 1-21 of the second dosing cycle.

27. Use of lenalidomide in combination with mosunetuzumab for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about 45 mg, and
(c) the second dosing cycle further comprises about 10 mg lenalidomide to be orally administered daily on Days 1-21 of the second dosing cycle.

28. Use of mosunetuzumab in the manufacture of a medicament for use in combination with lenalidomide for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about mg, and
(c) the second dosing cycle further comprises about 10 mg lenalidomide to be orally administered daily on Days 1-21 of the second dosing cycle.

29. Use of lenalidomide in the manufacture of a medicament for use in combination with mosunetuzumab for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about mg, and
(c) the second dosing cycle further comprises about 10 mg lenalidomide to be orally administered daily on Days 1-21 of the second dosing cycle.

30. A method of treating a subject having a previously untreated FL, the method comprising administering to the subject mosunetuzumab and lenalidomide according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab subcutaneously administered on Day 1 of the second dosing cycle, wherein the C2D1 is about 45 mg, and
(c) the second dosing cycle further comprises orally administering about 20 mg lenalidomide daily on Days 1-21 of the second dosing cycle.

31. Mosunetuzumab for use in combination with lenalidomide for treating a subject having a previously untreated F), wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about mg, and
(c) the second dosing cycle further comprises about 20 mg lenalidomide to be orally administered daily on Days 1-21 of the second dosing cycle.

32. Lenalidomide for use in combination with mosunetuzumab for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about 45 mg, and
(c) the second dosing cycle further comprises about 20 mg lenalidomide to be orally administered daily on Days 1-21 of the second dosing cycle.

33. Use of mosunetuzumab in combination with lenalidomide for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about mg, and
(c) the second dosing cycle further comprises about 20 mg lenalidomide to be orally administered daily on Days 1-21 of the second dosing cycle.

34. Use of lenalidomide in combination with mosunetuzumab for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about mg, and
(c) the second dosing cycle further comprises about 20 mg lenalidomide to be orally administered daily on Days 1-21 of the second dosing cycle.

35. Use of mosunetuzumab in the manufacture of a medicament for use in combination with lenalidomide for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about 45 mg, and
(c) the second dosing cycle further comprises about 20 mg lenalidomide to be orally administered daily on Days 1-21 of the second dosing cycle.

36. Use of lenalidomide in the manufacture of a medicament for use in combination with mosunetuzumab for treating a subject having a previously untreated FL, wherein mosunetuzumab and lenalidomide are to be administered to the subject according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab to be subcutaneously administered on Day 1 of the second dosing cycle, wherein the C2D1 is about mg, and
(c) the second dosing cycle further comprises about 20 mg lenalidomide to be orally administered daily on Days 1-21 of the second dosing cycle.

37. A method of treating a subject having a previously untreated FL, the method comprising administering to the subject mosunetuzumab and lenalidomide according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab subcutaneously administered on Day 1 of each dosing cycle, wherein each single dose C2D1-C12D1 is about 45 mg, and
(c) the second to twelfth dosing cycles each further comprises orally administering about 10 mg lenalidomide daily on Days 1-21 of each dosing cycle.

38. Mosunetuzumab for use in combination with lenalidomide for treating a subject having a previously untreated F), wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab to be subcutaneously administered on Day 1 of each dosing cycle, wherein each single dose C2D1-C12D1 is about 45 mg, and
(c) the second to twelfth dosing cycles each further comprises about 10 mg lenalidomide to be orally administered daily on Days 1-21 of each dosing cycle.

39. Lenalidomide for use in combination with mosunetuzumab for treating a subject having a previously untreated FL, wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab to be subcutaneously administered on Day 1 of each dosing cycle, wherein each single dose C2D1-C12D1 is about 45 mg, and
(c) the second to twelfth dosing cycles each further comprises about 10 mg lenalidomide to be orally administered daily on Days 1-21 of each dosing cycle.

40. Use of mosunetuzumab in combination with lenalidomide for treating a subject having a previously untreated FL, wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab to be subcutaneously administered on Day 1 of each dosing cycle, wherein each single dose C2D1-C12D1 is about 45 mg, and
(c) the second to twelfth dosing cycles each further comprises about 10 mg lenalidomide to be orally administered daily on Days 1-21 of each dosing cycle.

41. Use of lenalidomide in combination with mosunetuzumab for treating a subject having a previously untreated FL, wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab to be subcutaneously administered on Day 1 of each dosing cycle, wherein each single dose C2D1-C12D1 is about 45 mg, and
(c) the second to twelfth dosing cycles each further comprises about 10 mg lenalidomide to be orally administered daily on Days 1-21 of each dosing cycle.

42. Use of mosunetuzumab in the manufacture of a medicament for use in combination with lenalidomide for treating a subject having a previously untreated FL, wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab to be subcutaneously administered on Day 1 of each dosing cycle, wherein each single dose C2D1-C12D1 is about 45 mg, and
(c) the second to twelfth dosing cycles each further comprises about 10 mg lenalidomide to be orally administered daily on Days 1-21 of each dosing cycle.

43. Use of lenalidomide in the manufacture of a medicament for use in combination with mosunetuzumab for treating a subject having a previously untreated FL, wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab to be subcutaneously administered on Day 1 of each dosing cycle, wherein each single dose C2D1-C12D1 is about 45 mg, and
(c) the second to twelfth dosing cycles each further comprises about 10 mg lenalidomide to be orally administered daily on Days 1-21 of each dosing cycle.

44. A method of treating a subject having a previously untreated FL, the method comprising administering to the subject mosunetuzumab and lenalidomide according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab subcutaneously administered on Day 1 of each dosing cycle, wherein each single dose C2D1-C12D1 is about 45 mg, and
(c) the second to twelfth dosing cycles each further comprises orally administering about 20 mg lenalidomide daily on Days 1-21 of each dosing cycle.

45. Mosunetuzumab for use in combination with lenalidomide for treating a subject having a previously untreated F), wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab to be subcutaneously administered on Day 1 of each dosing cycle, wherein each single dose C2D1-C12D1 is about 45 mg, and
(c) the second to twelfth dosing cycles each further comprises about 20 mg lenalidomide to be orally administered daily on Days 1-21 of each dosing cycle.

46. Lenalidomide for use in combination with mosunetuzumab for treating a subject having a previously untreated FL, wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab to be subcutaneously administered on Day 1 of each dosing cycle, wherein each single dose C2D1-C12D1 is about 45 mg, and
(c) the second to twelfth dosing cycles each further comprises about 20 mg lenalidomide to be orally administered daily on Days 1-21 of each dosing cycle.

47. Use of mosunetuzumab in combination with lenalidomide for treating a subject having a previously untreated FL, wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab to be subcutaneously administered on Day 1 of each dosing cycle, wherein each single dose C2D1-C12D1 is about 45 mg, and
(c) the second to twelfth dosing cycles each further comprises about 20 mg lenalidomide to be orally administered daily on Days 1-21 of each dosing cycle.

48. Use of lenalidomide in combination with mosunetuzumab for treating a subject having a previously untreated FL, wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab to be subcutaneously administered on Day 1 of each dosing cycle, wherein each single dose C2D1-C12D1 is about 45 mg, and
(c) the second to twelfth dosing cycles each further comprises about 20 mg lenalidomide to be orally administered daily on Days 1-21 of each dosing cycle.

49. Use of mosunetuzumab in the manufacture of a medicament for use in combination with lenalidomide for treating a subject having a previously untreated FL, wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab to be subcutaneously administered on Day 1 of each dosing cycle, wherein each single dose C2D1-C12D1 is about 45 mg, and
(c) the second to twelfth dosing cycles each further comprises about 20 mg lenalidomide to be orally administered daily on Days 1-21 of each dosing cycle.

50. Use of lenalidomide in the manufacture of a medicament for use in combination with mosunetuzumab for treating a subject having a previously untreated FL, wherein mosunetuzumab and lenalidomide are to be administered to the subject according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab to be subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab to be subcutaneously administered on Day 1 of each dosing cycle, wherein each single dose C2D1-C12D1 is about 45 mg, and
(c) the second to twelfth dosing cycles each further comprises about 20 mg lenalidomide to be orally administered daily on Days 1-21 of each dosing cycle.

51. The method, mosunetuzumab for use, lenalidomide for use, or use of any one of embodiments 1-50, wherein the FL is histologically documented as Grade 1, 2, or 3a, but not 3b according to the World Health Organization classification of lymphoid neoplasms (as referenced in Swerdlow S H, et al. Blood 2016; 127:2375-90).

52. The method, mosunetuzumab for use, lenalidomide for use, or use of any one of embodiments 1-51, wherein the subject has been previously determined based on the Groupe d'Etude des Lymphomes Folliculaires (GELF) criteria (Brice et al. J Clin Oncol. 15(3):1110-1117, 1997) to be in need of systemic therapy to treat the previously untreated FL.

53. The method, mosunetuzumab for use, lenalidomide for use, or use of any one of embodiments 1-52, wherein the first dosing cycle further comprises administration of a corticosteroid.

54. The method, mosunetuzumab for use, lenalidomide for use, or use of any one of embodiments 1-53, wherein the second dosing cycle further comprises administration of a corticosteroid.

The method, mosunetuzumab for use, lenalidomide for use, or use of any one of embodiments 8-54, wherein any of the one or more additional dosing cycles comprises administration of a corticosteroid.

56. The method, mosunetuzumab for use, lenalidomide for use, or use of any one of embodiments 53-55, wherein a single dose of the corticosteroid is administered or is to be administered to the subject prior to the administration of any dose of mosunetuzumab.

57. The method, mosunetuzumab for use, lenalidomide for use, or use of any one of embodiments 53-56, wherein the corticosteroid comprises dexamethasone or methylprednisolone.

58. The method, mosunetuzumab for use, lenalidomide for use, or use of any one of embodiments 53-57, wherein the corticosteroid is administered or is to be administered intravenously or orally.

59. The method, mosunetuzumab for use, lenalidomide for use, or use of any one of embodiments 53-58, wherein the corticosteroid comprises dexamethasone and is administered or is to be administered at a dose of about 20 mg.

60. The method, mosunetuzumab for use, lenalidomide for use, or use of any one of embodiments 53-27, wherein the corticosteroid comprises methylprednisolone and is administered or is to be administered at a dose of about 58 mg.

61. The method, mosunetuzumab for use, lenalidomide for use, or use of any one of embodiments 1-60, wherein the first dosing cycle further comprises administration of an antihistamine.

62. The method, mosunetuzumab for use, lenalidomide for use, or use of any one of embodiments 1-61, wherein the second dosing cycle further comprises administration of an antihistamine.

63. The method, mosunetuzumab for use, lenalidomide for use, or use of any one of embodiments 8-62, wherein any of the one or more additional dosing cycles comprises administration of an antihistamine.

64. The method, mosunetuzumab for use, lenalidomide for use, or use of any one of embodiments 61-63, wherein a single dose of the antihistamine is administered or is to be administered to the subject prior to the administration of any dose of mosunetuzumab.

65. The method, mosunetuzumab for use, lenalidomide for use, or use of embodiment 64, wherein the antihistamine is administered or is to be administered to the subject at least 30 minutes prior to the administration of any dose of mosunetuzumab.

66. The method, mosunetuzumab for use, lenalidomide for use, or use of any one of embodiments 61-65, wherein the antihistamine is administered or is to be administered orally or intravenously.

67. The method, mosunetuzumab for use, lenalidomide for use, or use of any one of embodiments 61-66, wherein the antihistamine comprises diphenhydramine hydrochloride and is administered or is to be administered at a dose of about 50-100 mg.

68. The method, mosunetuzumab for use, lenalidomide for use, or use of any one of embodiments 1-67, wherein the first dosing cycle further comprises administration of an anti-pyretic.

69. The method, mosunetuzumab for use, lenalidomide for use, or use of any one of embodiments 1-68, wherein the second dosing cycle further comprises administration of an anti-pyretic.

70. The method, mosunetuzumab for use, lenalidomide for use, or use of any one of embodiments 8-69, wherein any of the one or more additional dosing cycles comprises administration of an anti-pyretic.

71. The method, mosunetuzumab for use, lenalidomide for use, or use of any one of embodiments 68-70, wherein a single dose of the anti-pyretic is administered or is to be administered to the subject prior to the administration of any dose of mosunetuzumab.

72. The method, mosunetuzumab for use, lenalidomide for use, or use of embodiment 71, wherein the anti-pyretic is administered or is to be administered to the subject at least 30 minutes prior to the administration of any dose of mosunetuzumab.

73. The method, mosunetuzumab for use, lenalidomide for use, or use of any one of embodiments 68-72, wherein the anti-pyretic is administered or is to be administered orally.

74. The method, mosunetuzumab for use, lenalidomide for use, or use of any one of embodiments 68-73, wherein the anti-pyretic comprises acetaminophen and is administered or is to be administered at a dose of about 500-1000 mg.

75. The method, mosunetuzumab for use, lenalidomide for use, or use of any one of embodiments 1-74, wherein the first dosing cycle further comprises administration of an initial dose of a prophylactic agent against tumor lysis syndrome (TLS).

76. The method, mosunetuzumab for use, lenalidomide for use, or use of any one of embodiments 1-75, wherein the second dosing cycle further comprises administration of an initial dose of a prophylactic agent against TLS.

77. The method, mosunetuzumab for use, lenalidomide for use, or use of any one of embodiments 8-76, wherein any of the one or more additional dosing cycles comprises administration of an initial dose of a prophylactic agent against TLS.

78. The method, mosunetuzumab for use, lenalidomide for use, or use of any one of embodiments 75-77, wherein the initial dose of the prophylactic agent against TLS is administered or is to be administered to the subject prior to the administration of any dose of mosunetuzumab.

79. The method, mosunetuzumab for use, lenalidomide for use, or use of any one of embodiments 75-78, wherein the prophylactic agent against TLS comprises allopurinol.

80. The method, mosunetuzumab for use, lenalidomide for use, or use of embodiment 79, wherein the initial dose of allopurinol is administered or is to be administered about 72 hours prior to the administration of any dose of mosunetuzumab.

81. The method, mosunetuzumab for use, lenalidomide for use, or use of embodiment 80, wherein additional single doses of allopurinol are administered or are to be administered daily for 6-10 days after the administration of the initial dose.

82. The method, mosunetuzumab for use, lenalidomide for use, or use of any one of embodiments 79-81, wherein the initial dose of allopurinol is about 300 mg.

83. The method, mosunetuzumab for use, lenalidomide for use, or use of embodiment 81 or 82, wherein each additional single dose of allopurinol is about 300 mg.

84. The method, mosunetuzumab for use, lenalidomide for use, or use of any one of embodiments 79-83, wherein allopurinol is administered or is to be administered orally.

85. The method, mosunetuzumab for use, lenalidomide for use, or use of any one of embodiments 75-78, wherein the prophylactic agent against TLS comprises rasburicase.

86. The method, mosunetuzumab for use, lenalidomide for use, or use of embodiment 85, wherein the initial dose of rasburicase is administered or is to be administered about 30 minutes prior to the administration of any dose of mosunetuzumab.

87. The method, mosunetuzumab for use, lenalidomide for use, or use of embodiment 86, wherein additional single doses of rasburicase are administered or are to be administered daily for 1-5 days after the administration of the initial dose.

88. The method, mosunetuzumab for use, lenalidomide for use, or use of any one of embodiments 85-87, wherein the initial dose of rasburicase is about 0.2 mg/kg.

89. The method, mosunetuzumab for use, lenalidomide for use, or use of embodiment 87 or 88, wherein each additional single dose of rasburicase is about 0.2 mg/kg.

90. The method, mosunetuzumab for use, lenalidomide for use, or use of any one of embodiments 85-89, wherein rasburicase is administered or is to be administered intravenously.

91. The method, mosunetuzumab for use, lenalidomide for use, or use of any one of embodiments 1-90, wherein the first dosing cycle is a three-dose dosing cycle.

92. The method, mosunetuzumab for use, lenalidomide for use, or use of any one of embodiments 1-91, wherein the subject is human.

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating a subject having a previously untreated follicular lymphoma (FL), the method comprising administering to the subject mosunetuzumab and lenalidomide according to a dosing regimen comprising a first 21-day dosing cycle and a second 28-day dosing cycle, wherein:
(a) the first dosing cycle comprises a first dose (C1D1), a second dose (C1D2), and a third dose (C1D3) of mosunetuzumab subcutaneously administered on Days 1, 8, and 15, respectively, of the first dosing cycle, wherein the C1D1 is about 5 mg, the C1D2 is about 45 mg, and the C1D3 is about 45 mg,
(b) the second dosing cycle comprises a single dose (C2D1) of mosunetuzumab subcutaneously administered on Day 1 of the second dosing cycle, wherein the C2D1 is about 45 mg, and
(c) the second dosing cycle further comprises orally administering between about 5 mg and about 20 mg lenalidomide daily on Days 1-21 of the second dosing cycle.

2. The method of claim 1, wherein:
(a) the dosing regimen comprises one or more additional dosing cycles;
(b) lenalidomide is not administered during the first dosing cycle;
(c) lenalidomide is not administered on the last 7 days of any dosing cycle comprising administration of lenalidomide;
(d) lenalidomide is administered at a dose of about 10 mg or about 20 mg;
(e) the FL is histologically documented as Grade 1, 2, or 3a, but not 3b according to the World Health Organization classification of lymphoid neoplasms (as referenced in Swerdlow S H, et al. Blood 2016; 127:2375-90);
(f) the subject has been previously determined based on the Groupe d'Etude des Lymphomes Folliculaires (GELF) criteria (Brice et al. J Clin Oncol. 15(3):1110-1117, 1997) to be in need of systemic therapy to treat the previously untreated FL;
(g) the first dosing cycle further comprises administration of a corticosteroid, an antihistamine, an anti-pyretic and/or an initial dose of a prophylactic agent against tumor lysis syndrome (TLS); and/or
(h) the second dosing cycle further comprises administration of a corticosteroid, an antihistamine, an anti-pyretic, and/or an initial dose of a prophylactic agent against TLS.

3. The method of claim 2, wherein:
(a) the dosing regimen comprises one to ten additional dosing cycles;
(b) the length of each of the one or more additional dosing cycles is about 28 days;
(c) each of the one or more additional dosing cycles comprises an additional single dose of mosunetuzumab;
(d) lenalidomide is orally administered during any of the one or more additional dosing cycles; and/or
(e) any of the one or more additional dosing cycles comprises administration of a corticosteroid, an antihistamine, an anti-pyretic, and/or an initial dose of a prophylactic agent against TLS.

4. The method of claim 3, wherein:
(a) the dosing regimen comprises ten additional dosing cycles;
(b) the additional single dose of mosunetuzumab is about 45 mg;
(c) the method comprises subcutaneously administering to the subject each additional single dose of mosunetuzumab on Day 1 of each of the one or more additional dosing cycles; and/or
(d) is orally administered on Days 1-21 of each of the additional dosing cycles comprising administration of lenalidomide.

5-17. (canceled)

18. A method of treating a subject having a previously untreated FL, the method comprising administering to the subject mosunetuzumab and lenalidomide according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab subcutaneously administered on Day 1 of each dosing cycle, wherein each single dose C2D1-C12D1 is about 45 mg, and
(c) the second to twelfth dosing cycles each further comprises orally administering about 10 mg lenalidomide daily on Days 1-21 of each dosing cycle.

19. A method of treating a subject having a previously untreated FL, the method comprising administering to the subject mosunetuzumab and lenalidomide according to a dosing regimen comprising a first 21-day dosing cycle and eleven subsequent 28-day dosing cycles, wherein:
(a) the first dosing cycle comprises a first dose (C1D1), a second dose (C1D2), and a third dose (C1D3) of mosunetuzumab subcutaneously administered on Days 1, 8, and 15, respectively, of the first dosing cycle, wherein the C1D1 is about 5 mg, the C1D2 is about 45 mg, and the C1D3 is about 45 mg,
(b) the second to twelfth dosing cycles each comprises a single dose (C2D1-C12D1) of mosunetuzumab subcutaneously administered on Day 1 of each dosing cycle, wherein each single dose C2D1-C12D1 is about 45 mg, and
(c) the second to twelfth dosing cycles each further comprises orally administering about 20 mg lenalidomide daily on Days 1-21 of each dosing cycle.

20. The method of claim 18, wherein:
(a) the FL is histologically documented as Grade 1, 2, or 3a, but not 3b according to the World Health Organization classification of lymphoid neoplasms (as referenced in Swerdlow S H, et al. Blood 2016; 127:2375-90);
(b) the subject has been previously determined based on the Groupe d'Etude des Lymphomes Folliculaires (GELF) criteria (Brice et al. J Clin Oncol. 15(3):1110-1117, 1997) to be in need of systemic therapy to treat the previously untreated FL;
(c) the first dosing cycle further comprises administration of a corticosteroid, an antihistamine, an anti-pyretic, and/or an initial dose of a prophylactic agent against TLS; and/or
(d) each of the second to twelfth dosing cycles further comprises administration of a corticosteroid, an antihistamine, an anti-pyretic, and/or an initial dose of a prophylactic agent against TLS.

21-24. (canceled)

25. The method of claim 2, wherein:
(a) a single dose of the corticosteroid is administered to the subject prior to the administration of any dose of mosunetuzumab;
(b) the corticosteroid comprises dexamethasone or methylprednisolone;
(c) the corticosteroid is administered intravenously or orally;
(d) a single dose of the antihistamine is administered to the subject prior to the administration of any dose of mosunetuzumab;
(e) the antihistamine is administered orally or intravenously;
(f) a single dose of the anti-pyretic is administered to the subject prior to the administration of any dose of mosunetuzumab;
(g) the anti-pyretic is administered orally;
(h) the initial dose of the prophylactic agent against TLS is administered to the subject prior to the administration of any dose of mosunetuzumab; and/or
(i) the prophylactic agent against TLS comprises allopurinol and/or rasburicase.

26-27. (canceled)

28. The method of claim 25, wherein:
(a) the corticosteroid comprises dexamethasone and is administered at a dose of about 20 mg;
(b) the corticosteroid comprises methylprednisolone and is administered at a dose of about mg;
(c) the antihistamine is administered to the subject at least 30 minutes prior to the administration of any dose of mosunetuzumab;
(d) the antihistamine comprises diphenhydramine hydrochloride and is administered at a dose of about 50-100 mg;
(e) the anti-pyretic is administered to the subject at least 30 minutes prior to the administration of any dose of mosunetuzumab;
(f) the anti-pyretic comprises acetaminophen and is administered at a dose of about 500-1000 mg;
(g) the initial dose of allopurinol is administered about 72 hours prior to the administration of any dose of mosunetuzumab;
(h) the initial dose of allopurinol is about 300 mg;
(i) allopurinol is administered orally;
(j) the initial dose of rasburicase is administered about 30 minutes prior to the administration of any dose of mosunetuzumab;
(k) the initial dose of rasburicase is about 0.2 mg/kg; and/or
(l) rasburicase is administered intravenously.

29-49. (canceled)

50. The method of claim 28, wherein:
(a) additional single doses of allopurinol are administered daily for 6-10 days after the administration of the initial dose; and/or
(b) additional single doses of rasburicase are administered daily for 1-5 days after the administration of the initial dose.

51. (canceled)

52. The method of claim 50, wherein:
(a) each additional single dose of allopurinol is about 300 mg; and/or
(b) each additional single dose of rasburicase is about 0.2 mg/kg.

53-59. (canceled)

60. The method of claim 20, wherein:
(a) a single dose of the corticosteroid is administered to the subject prior to the administration of any dose of mosunetuzumab;
(b) the corticosteroid comprises dexamethasone or methylprednisolone;
(c) the corticosteroid is administered intravenously or orally;
(d) a single dose of the antihistamine is administered to the subject prior to the administration of any dose of mosunetuzumab;
(e) the antihistamine is administered orally or intravenously;
(f) a single dose of the anti-pyretic is administered to the subject prior to the administration of any dose of mosunetuzumab;
(g) the anti-pyretic is administered orally;
(h) the initial dose of the prophylactic agent against TLS is administered to the subject prior to the administration of any dose of mosunetuzumab; and/or
(i) the prophylactic agent against TLS comprises allopurinol and/or rasburicase.

61. The method of claim 60, wherein:
(a) the corticosteroid comprises dexamethasone and is administered at a dose of about 20 mg;
(b) the corticosteroid comprises methylprednisolone and is administered at a dose of about mg;
(c) the antihistamine is administered to the subject at least 30 minutes prior to the administration of any dose of mosunetuzumab;
(d) the antihistamine comprises diphenhydramine hydrochloride and is administered at a dose of about 50-100 mg;
(e) the anti-pyretic is administered to the subject at least 30 minutes prior to the administration of any dose of mosunetuzumab;
(f) the anti-pyretic comprises acetaminophen and is administered at a dose of about 500-1000 mg;
(g) the initial dose of allopurinol is administered about 72 hours prior to the administration of any dose of mosunetuzumab;
(h) the initial dose of allopurinol is about 300 mg;
(i) allopurinol is administered orally;
(j) the initial dose of rasburicase is administered about 30 minutes prior to the administration of any dose of mosunetuzumab;
(k) the initial dose of rasburicase is about 0.2 mg/kg; and/or
(l) rasburicase is administered intravenously.

62. The method of claim 61, wherein:
(a) additional single doses of allopurinol are administered daily for 6-10 days after the administration of the initial dose; and/or
(b) additional single doses of rasburicase are administered daily for 1-5 days after the administration of the initial dose.

63. The method of claim 62, wherein:
(a) each additional single dose of allopurinol is about 300 mg; and/or
(b) each additional single dose of rasburicase is about 0.2 mg/kg.

64. The method of claim 19, wherein:
(a) the FL is histologically documented as Grade 1, 2, or 3a, but not 3b according to the World Health Organization classification of lymphoid neoplasms (as referenced in Swerdlow S H, et al. Blood 2016; 127:2375-90);
(b) the subject has been previously determined based on the Groupe d'Etude des Lymphomes Folliculaires (GELF) criteria (Brice et al. J Clin Oncol. 15(3):1110-1117, 1997) to be in need of systemic therapy to treat the previously untreated FL;
(c) the first dosing cycle further comprises administration of a corticosteroid, an antihistamine, an anti-pyretic, and/or an initial dose of a prophylactic agent against TLS; and/or
(d) each of the second to twelfth dosing cycles further comprises administration of a corticosteroid, an antihistamine, an anti-pyretic, and/or an initial dose of a prophylactic agent against TLS.

65. The method of claim 64, wherein:
(a) a single dose of the corticosteroid is administered to the subject prior to the administration of any dose of mosunetuzumab;
(b) the corticosteroid comprises dexamethasone or methylprednisolone;
(c) the corticosteroid is administered intravenously or orally;
(d) a single dose of the antihistamine is administered to the subject prior to the administration of any dose of mosunetuzumab;
(e) the antihistamine is administered orally or intravenously;
(f) a single dose of the anti-pyretic is administered to the subject prior to the administration of any dose of mosunetuzumab;
(g) the anti-pyretic is administered orally;
(h) the initial dose of the prophylactic agent against TLS is administered to the subject prior to the administration of any dose of mosunetuzumab; and/or
(i) the prophylactic agent against TLS comprises allopurinol and/or rasburicase.

66. The method of claim 65, wherein:
(a) the corticosteroid comprises dexamethasone and is administered at a dose of about 20 mg;
(b) the corticosteroid comprises methylprednisolone and is administered at a dose of about mg;
(c) the antihistamine is administered to the subject at least 30 minutes prior to the administration of any dose of mosunetuzumab;
(d) the antihistamine comprises diphenhydramine hydrochloride and is administered at a dose of about 50-100 mg;
(e) the anti-pyretic is administered to the subject at least 30 minutes prior to the administration of any dose of mosunetuzumab;
(f) the anti-pyretic comprises acetaminophen and is administered at a dose of about 500-1000 mg;
(g) the initial dose of allopurinol is administered about 72 hours prior to the administration of any dose of mosunetuzumab;
(h) the initial dose of allopurinol is about 300 mg;
(i) allopurinol is administered orally;
(j) the initial dose of rasburicase is administered about 30 minutes prior to the administration of any dose of mosunetuzumab;
(k) the initial dose of rasburicase is about 0.2 mg/kg; and/or
(l) rasburicase is administered intravenously.

67. The method of claim 66, wherein:
(a) additional single doses of allopurinol are administered daily for 6-10 days after the administration of the initial dose; and/or
(b) additional single doses of rasburicase are administered daily for 1-5 days after the administration of the initial dose.

68. The method of claim 67, wherein:
(a) each additional single dose of allopurinol is about 300 mg; and/or
(b) each additional single dose of rasburicase is about 0.2 mg/kg.


SEQUENCE LISTING (36 sequences)

SEQ ID NO: 1 - protein, 10 aa, synthetic construct
GYTFTSYNMH

SEQ ID NO: 2 - protein, 17 aa, synthetic construct
AIYPGNGDTSYNQKFKG

SEQ ID NO: 3 - protein, 13 aa, synthetic construct
VVYYSNSYWYFDV

SEQ ID NO: 4 - protein, 10 aa, synthetic construct
RASSSVSYMH

SEQ ID NO: 5 - protein, 7 aa, synthetic construct
APSNLAS

SEQ ID NO: 6 - protein, 9 aa, synthetic construct
QQWSFNPPT

SEQ ID NO: 7 - protein, 122 aa, synthetic construct [sequence omitted]

SEQ ID NO: 8 - protein, 106 aa, synthetic construct [sequence omitted]

SEQ ID NO: 9 - protein, 5 aa, synthetic construct
NYYIH

SEQ ID NO: 10 - protein, 17 aa, synthetic construct
WIYPGDGNTKYNEKFKG

SEQ ID NO: 11 - protein, 10 aa, synthetic construct
DSYSNYYFDY

SEQ ID NO: 12 - protein, 17 aa, synthetic construct
KSSQSLLNSRTRKNYLA

SEQ ID NO: 13 - protein, 7 aa, synthetic construct
WASTRES

SEQ ID NO: 14 - protein, 8 aa, synthetic construct
TQSFILRT

SEQ ID NO: 15 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 16 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 17 - protein, 25 aa, synthetic construct [sequence omitted]

SEQ ID NO: 18 - protein, 14 aa, synthetic construct
WVRQAPGKGLEWVG

SEQ ID NO: 19 - protein, 32 aa, synthetic construct
RFTISVDKSKNTLYLQMNSLRAEDTAVYYCAR

SEQ ID NO: 20 - protein, 11 aa, synthetic construct
WGQGTLVTVSS

SEQ ID NO: 21 - protein, 23 aa, synthetic construct
DIQMTQSPSSLSASVGDRVTITC

SEQ ID NO: 22 - protein, 15 aa, synthetic construct
WYQQKPGKAPKPLIY

SEQ ID NO: 23 - protein, 32 aa, synthetic construct
GVPSRFSGSGSGTDFTLTISSLQPEDFATYYC

SEQ ID NO: 24 - protein, 10 aa, synthetic construct
FGQGTKVEIK

SEQ ID NO: 25 - protein, 30 aa, synthetic construct
EVQLVQSGAEVKKPGASVKVSCKASGYTFT

SEQ ID NO: 26 - protein, 14 aa, synthetic construct
WVRQAPGQGLEWIG

SEQ ID NO: 27 - protein, 32 aa, synthetic construct
RATLTADTSTSTAYLELSSLRSEDTAVYYCAR

SEQ ID NO: 28 - protein, 11 aa, synthetic construct
WGQGTLVTVSS

SEQ ID NO: 29 - protein, 23 aa, synthetic construct
DIVMTQSPDSLAVSLGERATINC

SEQ ID NO: 30 - protein, 15 aa, synthetic construct
WYQQKPGQPPKLLIY

SEQ ID NO: 31 - protein, 32 aa, synthetic construct
GVPDRFSGSGSGTDFTLTISSLQAEDVAVYYC

SEQ ID NO: 32 - protein, 10 aa, synthetic construct
FGQGTKVEIK

SEQ ID NO: 33 - protein, 452 aa, synthetic construct
EVQLVESGGGLVQPGGSLRLSCAASGYTFTSYNMHWVRQAPGKGLEWVGAIYPGNGDTSYNQKFKGRFTISVDKSKNTLYLQMNSLRAEDTAVYYCARVVYYSNSYWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 213 aa, synthetic construct
DIQMTQSPSSLSASVGDRVTITCRASSSVSYMHWYQQKPGKAPKPLIYAPSNLASGVPSRFSGSGSGTDFTLTISSLQPEDFATYYCQQWSFNPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 449 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 36 - protein, 219 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC